(19) United States
(12) Patent Application Publication
(10) Pub. No.: US 2009/0318976 A1
(43) Pub. Date: Dec. 24, 2009

(54) IMPLANTABLE BRACE FOR PROVIDING JOINT SUPPORT

(75) Inventors: Stefan Gabriel, Mattapoisett, MA (US); Anton G. Clifford, Mountain View, CA (US); David Lowe, Redwood City, CA (US); Mary O'Connell, Menlo Park, CA (US); Michael E. Landry, Austin, TX (US); Clinton N. Slone, San Francisco, CA (US); Alan C. Regala, Mountain View, CA (US); Kevin Sidow, Piedmont, CA (US); Eric Dremel, Seattle, WA (US); Joshua Makower, Los Altos, CA (US)

Correspondence Address: STEPTOE & JOHNSON - EXPLORAMED NC4, INC., 2121 AVENUE OF THE STARS, SUITE 2800, LOS ANGELES, CA, 90067, US

(73) Assignee: MOXIMED, INC., Mountain View, CA (US)

(21) Appl. No.: 12/488,260

(22) Filed: Jun. 19, 2009

Related U.S. Application Data
(63) Continuation-in-part of application No. 11/743,097, filed on May 1, 2007.
(60) Provisional application No. 61/132,629, filed on Jun. 19, 2008.

(51) Int. Cl. A61B 17/80; A61B 17/56

(52) U.S. Cl. 606/283; 606/286; 606/86 R

(57) ABSTRACT

Internal braces and methods of implanting same. A brace can be implanted on one side of a joint, or a pair of braces can be implanted, one on each opposite side of a joint. Each brace supports the joint over at least a portion of its range of motion. Distraction may be provided, or load sharing can be accomplished without distraction. Relative axial rotation of the bones connected by the brace may be permitted. One or more compliant members may be provided in the brace.

### CROSS-REFERENCES TO RELATED APPLICATIONS

This application is a continuation-in-part of U.S. application Ser. No. 11/743,097, filed May 1, 2007, the contents of which are incorporated by reference, and claims the benefit of Provisional Application Ser. No. 61/132,629, filed Jun. 19, 2008, the contents of which are incorporated by reference.

### BACKGROUND OF THE INVENTION

Both humans and other mammals belong to the subphylum known as vertebrata. The defining characteristic of a vertebrate is considered the backbone or spinal cord, a brain case, and an internal skeleton. In biology, the skeleton or skeletal system is the biological system providing physical support in living organisms. Skeletal systems are commonly divided into three types—external (an exoskeleton), internal (an endoskeleton), and fluid based (a hydrostatic skeleton).

An internal skeletal system consists of rigid (or semi-rigid) structures, within the body, moved by the muscular system. If the structures are mineralized or ossified, as they are in humans and other mammals, they are referred to as bones. Cartilage is another common component of skeletal systems, supporting and supplementing the skeleton. The human ear and nose are shaped by cartilage. Some organisms have a skeleton consisting entirely of cartilage and without any calcified bones at all, for example sharks. The bones or other rigid structures are connected by ligaments and connected to the muscular system via tendons.

A joint is the location at which two or more bones make contact. They are constructed to allow movement and provide mechanical support, and are classified structurally and functionally. Structural classification is determined by how the bones are connected to each other, while functional classification is determined by the degree of movement between the articulating bones. In practice, there is significant overlap between the two types of classifications.

There are three structural classifications of joints, namely fibrous or immovable joints, cartilaginous joints and synovial joints. Fibrous/immovable bones are connected by dense connective tissue, consisting mainly of collagen. The fibrous joints are further divided into three types: sutures which are found between bones of the skull; syndesmosis which are found between long bones of the body; and gomphosis which is a joint between the root of a tooth and the sockets in the maxilla or mandible.

Cartilaginous bones are connected entirely by cartilage (also known as “synchondroses”). Cartilaginous joints allow more movement between bones than a fibrous joint but less than the highly mobile synovial joint. Synovial joints have a space between the articulating bones for synovial fluid. This classification contains joints that are the most mobile of the three, and includes the knee and shoulder. These are further classified into ball and socket joints, condyloid joints, saddle joints, hinge joints, pivot joints, and gliding joints.

Joints can also be classified functionally, by the degree of mobility they allow. Synarthrosis joints permit little or no mobility. They can be categorized by how the two bones are joined together. That is, synchrondoses are joints where the two bones are connected by a piece of cartilage. Synostoses are where two bones that are initially separated eventually fuse together as a child approaches adulthood. By contrast, amphiarthrosis joints permit slight mobility. The two bone surfaces at the joint are both covered in hyaline cartilage and joined by strands of fibrocartilage. Most amphiarthrosis joints are cartilaginous.

Finally, diarthrosis joints permit a variety of movements (e.g. flexion, adduction, pronation). Only synovial joints are diarthrodial and they can be divided into six classes: 1. ball and socket—such as the shoulder or the hip and femur; 2. hinge—such as the elbow; 3. pivot—such as the radius and ulna; 4. condyloidal (or ellipsoidal)—such as the wrist between radius and carps, or knee; 5. saddle—such as the joint between carpal thumbs and metacarpals; and 6. gliding—such as between the carpals.

Synovial joints (or diarthroses, or diarthroidal joints) are the most common and most moveable type of joints in the body. As with all other joints in the body, synovial joints achieve movement at the point of contact of the articulating bones. Structural and functional differences distinguish the synovial joints from the two other types of joints in the body, with the main structural difference being the existence of a cavity between the articulating bones and the occupation of a fluid in that cavity that aids movement. The whole of a diarthrosis is contained by a ligamentous sac, the joint capsule or articular capsule. The surfaces of the two bones at the joint are covered in cartilage. The thickness of the cartilage varies with each joint, and sometimes may be of uneven thickness. Articular cartilage is multi-layered. A thin superficial layer provides a smooth surface for the two bones to slide against each other. Of all the layers, it has the highest concentration of collagen and the lowest concentration of proteoglycans, making it very resistant to shear stresses. Deeper than that is an intermediate layer, which is mechanically designed to absorb shocks and distribute the load efficiently. The deepest layer is highly calcified, and anchors the articular cartilage to the bone. In joints where the two surfaces do not fit snugly together, a meniscus or multiple folds of fibro-cartilage within the joint correct the fit, ensuring stability and the optimal distribution of load forces. The synovium is a membrane that covers all the non-cartilaginous surfaces within the joint capsule. It secretes synovial fluid into the joint, which nourishes and lubricates the articular cartilage. The synovium is separated from the capsule by a layer of cellular tissue that contains blood vessels and nerves.

Cartilage is a type of dense connective tissue and as noted above, it forms a critical part of the functionality of a body joint. It is composed of collagenous fibers and/or elastin fibers, and cells called chondrocytes, all of which are embedded in a firm gel-like ground substance called the matrix. Articular cartilage is avascular (contains no blood vessels) and nutrients are diffused through the matrix. Cartilage serves several functions, including providing a framework upon which bone deposition can begin and supplying smooth surfaces for the movement of articulating bones. Cartilage is found in many places in the body including the joints, the rib cage, the ear, the nose, the bronchial tubes and between intervertebral discs. There are three main types of cartilage: hyaline, elastic and fibrocartilage.

Cancellous bone (also known as trabecular, or spongy) is a type of osseous tissue which also forms an important aspect of a body joint. Cancellous bone has a low density and strength but very high surface area, that fills the inner cavity of long bones. The external layer of cancellous bone contains red bone marrow where the production of blood cellular components (known as hematopoiesis) takes place. Cancellous bone is also where most of the arteries and veins of bone organs are found. The second type of osseous tissue is known as cortical bone, forming the hard outer layer of bone organs.

Various maladies can affect the joints, one of which is arthritis. Arthritis is a group of conditions where there is damage caused to the joints of the body. Arthritis is the leading cause of disability in people over the age of 65.

There are many forms of arthritis, each of which has a different cause. Rheumatoid arthritis and psoriatic arthritis are autoimmune diseases in which the body is attacking itself. Septic arthritis is caused by joint infection. Gouty arthritis is caused by deposition of uric acid crystals in the joint that results in subsequent inflammation. The most common form of arthritis, osteoarthritis is also known as degenerative joint disease and occurs following trauma to the joint, following an infection of the joint or simply as a result of aging.

Unfortunately, all arthritides feature pain. Patterns of pain differ among the arthritides and the location. Rheumatoid arthritis is generally worse in the morning; in the early stages, patients often do not have symptoms following their morning shower.

Osteoarthritis (OA, also known as degenerative arthritis or degenerative joint disease, and sometimes referred to as “arthrosis” or “osteoarthrosis” or in more colloquial terms “wear and tear”), is a condition in which low-grade inflammation results in pain in the joints, caused by wearing of the cartilage that covers and acts as a cushion inside joints. As the bone surfaces become less well protected by cartilage, the individual experiences pain upon weight bearing, including walking and standing. Due to decreased movement because of the pain, regional muscles may atrophy, and ligaments may become more lax. OA is the most common form of arthritis.

The main symptom of osteoarthritis is chronic pain, causing loss of mobility and often stiffness. “Pain” is generally described as a sharp ache in the joint, or a burning sensation in the associated muscles and tendons. OA can cause a crackling noise (called “crepitus”) when the affected joint is moved or touched, and individuals may experience muscle spasm and contractions in the tendons. Occasionally, the joints may also be filled with fluid. Humid weather increases the pain in many individuals.

OA commonly affects the hands, feet, spine, and the large weight-bearing joints, such as the hips and knees, although in theory, any joint in the body can be affected. As OA progresses, the affected joints appear larger, are stiff and painful, and usually feel worse, the more they are used and loaded throughout the day, thus distinguishing it from rheumatoid arthritis. With progression in OA, cartilage loses its viscoelastic properties and its ability to absorb load.

Generally speaking, the process of clinical detectable osteoarthritis is irreversible, and typical treatment consists of medication or other interventions that can reduce the pain of OA and thereby improve the function of the joint. According to an article entitled Surgical approaches for osteoarthritis by Klaus-Peter Günther, MD, over recent decades, a variety of surgical procedures have been developed with the aim of decreasing or eliminating pain and improving function in patients with advanced osteoarthritis (OA). The different approaches include preservation or restoration of articular surfaces, total joint replacement with artificial implants, and arthrodeses.

Arthrodeses are described as being reasonable alternatives for treating OA of small hand and foot joints as well as degenerative disorders of the spine, but were deemed to be rarely indicated in large weight-bearing joints such as the knee due to functional impairment of gait, cosmetic problems and further side-effects. Total joint replacement was characterized as an extremely effective treatment for severe joint disease. Moreover, recently developed joint-preserving treatment modalities were identified as having a potential to stimulate the formation of a new articular surface in the future. However, it was concluded that such techniques do not presently predictably restore a durable articular surface to an osteoarthritic joint. Thus, the correction of mechanical abnormalities by osteotomy and joint debridement are still considered as treatment options in many patients. Moreover, patients with limb malalignment, instability and intra-articular causes of mechanical dysfunction can benefit from an osteotomy to provide pain relief. The goal being the transfer of weight-bearing forces from arthritic portions to healthier locations of a joint.

Joint replacement is one of the most common and successful operations in modern orthopedic surgery. It consists of replacing painful, arthritic, worn or diseased parts of the joint with artificial surfaces shaped in such a way as to allow joint movement. Such procedures are a last resort treatment as they are highly invasive, require substantial periods of recovery and are irreversible. Joint replacement is sometimes called total joint replacement indicating that all joint surfaces are replaced. This contrasts with hemiarthroplasty (half arthroplasty) in which only one bone's joint surface is replaced and unincompartmental arthroplasty in which both surfaces of the knee, for example, are replaced but only on the inner or outer sides, not both. Thus, arthroplasty as a general term, is an operative procedure of orthopedic surgery performed, in which the arthritic or dysfunctional joint surface is replaced with something better. These procedures are also characterized by relatively long recovery times and their highly invasive procedures. The currently available therapies are not chondro-protective. Previously, a popular form of arthroplasty was interpositional arthroplasty with interposition of some other tissue like skin, muscle or tendon to keep inflammatory surfaces apart or excisional arthroplasty in which the joint surface and bone was removed leaving scar tissue to fill in the gap. Other forms of arthroplasty include resection(al) arthroplasty, resurfacing arthroplasty, mold arthroplasty, cup arthroplasty, silicone replacement arthroplasty, etc. Osteotomy to restore or modify joint congruity is also an arthroplasty.

Osteotomy is a related surgical procedure involving cutting of bone to improve alignment. The goal of osteotomy is to relieve pain by equalizing forces across the joint as well as increase the lifespan of the joint. This procedure is often used in younger, more active or heavier patients. High tibial osteotomy (HTO) is associated with a decrease in pain and improved function. However, HTO does not address ligamentous instability—only mechanical alignment. HTO is associated with good early results, but results deteriorate over time.

Certain other approaches to treating osteoarthritis contemplate external devices such as braces or fixators which limit the motion of the bones at a joint or apply cross-loads at a joint to shift load from one side of the joint to the other. Several of these approaches have had some success in alleviating pain but suffer from patient compliance or lack an ability to facilitate and support the natural motion and function of the diseased joint. Notably, the motion of bones forming a joint can be as distinctive as a finger print, and thus, each individual has his or her own unique set of problems to address. Therefore, mechanical approaches to treating osteoarthritis have had limited applications.

Load-induced pain in joints is a problem that occurs not only with individuals suffering from osteoarthritis, but with individuals having other types of joint diseases or injuries. Load-induced pain may be experienced as an increase in pain as the joint undergoes loading during normal use or may be experienced in a joint in which the individual does not experience pain when the joint is unloaded, but experiences pain over all or a portion of the pathway over which joint components interact with one another over the joint's range of motion. Pain levels may vary over different portion of the range of motion and may depend upon varying amounts of load born by the joint.

Temporary distraction of a joint has, in some cases been reported to allow healing/reconstruction of damaged cartilage that would normally carry loads when using the joint when not distracted. After a period of healing, in some instances about three to six months, the distraction is removed and improvements in the condition and functionality of the cartilage have been reported. Unloading and/or distracting a joint in these instances has allowed at least partial normalization of damaged cartilage.

There is a continuing need for treatment of joint pain by one or more implantable devices that address both joint movement and varying loads experienced by an articulating joint. There is further a need for improved implantable devices that distract an articulating joint as at least part of a treatment strategy for relieving pain.

The present invention satisfies these and other needs.

### SUMMARY OF THE INVENTION

The present invention provides internal braces and methods of implanting the same.

An internal brace for providing support to a joint is provided that includes a first component for attachment to a distal end portion of a first bone of a patient, the first component including a first upper portion configured to be fixed to the first bone and a first lower portion tapering from the first upper portion and including a first bearing surface; a second component for attachment to a proximal end portion of a second bone of the patient, wherein a joint is formed between the distal end portion of the first bone and the proximal end portion of the second bone, the second component including a second lower portion configured to be fixed to the second bone and a second upper portion tapering from the second lower portion and including a second bearing surface; wherein the first and second bearing surfaces are configured to allow relative rotation between the first and second bones and to allow at least one of: relative translation between said first and second bones along a direction; and at least a second degree of freedom of relative rotation between the first and second bones.

In at least one embodiment, the first and second bearing surfaces are configured to allow relative translation along an anterior-posterior direction.

In at least one embodiment, the first and second bearing surfaces articulate against one another.

In at least one embodiment, the first and second bearing surfaces each articulate with a third bearing member.

In at least one embodiment, the brace is configured to distract at least one side of the joint, so that the at least one side does not bear a load during at least some motions of the joint.

In at least one embodiment, the brace is configured to share load with at least one side of the joint, so that the at least one side of the joint bears a reduced load during at least some motions of the joint.

In at least one embodiment, the bearing surfaces of the brace support a load during only a portion of the full range of motion of the joint.

In at least one embodiment, the bearing surfaces of the brace are configured to support varying amounts of load over varying portions of the full range of motion of the joint.

In at least one embodiment, the brace is adjustable to vary at least one of: a location about which at least one of the bearing surfaces rotates; an amount of load taken up at different positions along the range of motion of the joint; an amount of distraction at different positions along the range of motion of the joint, and amount of compliance provided by the brace.

In at least one embodiment, the first lower portion and the second upper portion in combination form a wedge for distracting the joint.

In at least one embodiment, a pair of internal braces is adapted to be placed on both sides (i.e., one on the medial side and one on the lateral side) of a patient's knee joint.

In at least one embodiment, at least one compliant member is configured to allow axial movement between the first and second bones.

In at least one embodiment, the brace is configured to support a knee joint, wherein the first component comprises a femoral component and the first lower portion tapers outwardly into a condylar protrusion, the first bearing surface comprising a lower surface of the condylar protrusion, wherein the upper surface of the condylar protrusion is adapted to conform to the condyle, and wherein the first upper portion comprises a first inner surface configured to be attached to the femur and an outer surface that is external of the femur when the first inner surface is attached to the femur, and wherein the second component comprises a tibial component and the second upper portion tapers outwardly from the second lower portion into an upper tray comprising the second bearing surface for engaging the first bearing surface of the condylar protrusion, and wherein the second lower portion comprises a second inner surface configured to be attached to the tibia and a second lower portion outer surface that is external of the tibia when the second inner surface of the second lower portion is attached to the tibia.

In at least one embodiment, the femoral and tibial components are adapted to be attached to the medial side of the patient's knee, and the condylar protrusion and the upper tray in combination form a wedge adapted to fit into the meniscal space in the patient's medial joint.

In at least one embodiment, the femoral and tibial components are configured to be attached to the patient's femur and tibia, respectively, without substantially removing or replacing articular cartilage and with the first bearing surface engaging the second bearing surface, the condylar protrusion and the upper tray adapted to be positioned partially in the joint between the patient's intact femur and tibia and functioning to distract the joint.

A method for treating a joint is provided, including: providing an internal brace including a first component for attachment to a distal end portion of a first bone of a patient, the first component including a first upper portion configured to be fixed to the first bone and a first lower portion tapering from the first upper portion and including a first bearing surface, and a second component for attachment to a proximal end portion of a second bone of the patient, wherein the joint is formed between the distal end portion of the first bone and the proximal end portion of the second bone, the second component including a second lower portion configured to be fixed to the second bone and a second upper portion tapering from the second lower portion and including a second bearing surface; attaching the first upper portion of the first component to distal end portion of the patient's first bone; and attaching the second component to the proximal end portion of the patient's second bone such that the first bearing surface engages the second bearing surface without substantially removing or replacing articular cartilage in the joint, to support the joint, wherein the first and second bearing surfaces are configured to allow relative rotation between the first and second bones and to allow at least one of: relative translation between said first and second bones along a direction; and at least a second degree of freedom of relative rotation between the first and second bones.

In at least one embodiment, the first and second bearing surfaces are configured to allow relative translation along an anterior-posterior direction.

In at least one embodiment, one or more bones forming the joint which the brace is to be installed to are three-dimensionally scanned. From the scans of the one or more bones, one or more components of the brace can be custom designed to follow the contours of the one or more bones to which the component(s) is/are to be installed. If the components are for temporary implantation, they may be molded components, molded from suitable polymers. Alternatively, the components may be machined from titanium, chromium cobalt alloys, stainless steel, or other biocompatible materials suitable for making implantable braces.

In at least one embodiment, the brace is configured to support a knee joint, wherein the first component comprises a femoral component and the first lower portion tapers outwardly into a condylar protrusion, the first bearing surface comprising a lower surface of the condylar protrusion, wherein the upper surface of the condylar protrusion is adapted to conform to the condyle, and wherein the first upper portion comprises a first inner surface configured to be attached to the femur and an outer surface that is external of the femur when the first inner surface is attached to the femur, and wherein the second component comprises a tibial components and the second upper portion tapers outwardly from the second lower portion into an upper tray comprising the second bearing surface for engaging the first bearing surface of the condylar, and wherein the second lower portion comprises a second inner surface configured to be attached to the tibia and a second lower portion outer surface that is external of the tibia when the second inner surface of the second lower portion is attached to the tibia.

In at least one embodiment, the condylar protrusion and upper tray, in combination, form a wedge distracting the joint.

In at least one embodiment, the method further includes attaching an additional internal knee brace, whereby internal knee braces are attached to both the medial and lateral joints of the patient's knee.

A combination is provided, including an internal brace configured to be implanted on one side of a joint and an energy manipulation system configured to be implanted on an opposite side of the joint, The internal brace includes a first component for attachment to a distal end portion of a first bone of a patient, the first component including a first upper portion configured to be fixed to the first bone and a first lower portion tapering from the first upper portion and including a first bearing surface. The internal brace further includes a second component for attachment to a proximal end portion of a second bone of the patient, wherein the joint is formed between the distal end portion of the first bone and the proximal end portion of the second bone, and the second component includes a second lower portion configured to be fixed to the second bone and a second upper portion tapering from the second lower portion and including a second bearing surface. The first and second bearing surfaces are configured to allow relative rotation between the first and second bones.

The energy manipulation system includes a first attachment structure configured to be attached to the first bone, and a second attachment structure configured to be attached to the second bone. The energy manipulation system further includes an energy absorbing member attached to the first attachment structure and the second attachment structure.

In at least one embodiment, the first and second bearing surfaces are configured to further allow at least one of: relative translation between the first and second bones along a direction; and at least a second degree of freedom of relative rotation between the first and second bones.

These and other advantages and features of the invention will become apparent to those persons skilled in the art upon reading the details of the braces and methods as more fully described below.

### BRIEF DESCRIPTION OF THE DRAWINGS

FIGS. 1A-1B illustrate a perspective view and a sectional view of an embodiment of an internal brace for implantation in a patient to treat knee pain.

FIG. 1C is a sectional view of the brace of FIGS. 1A-1B implanted in the medial joint of a knee of a patient.

FIG. 1D illustrates a view of one example of a contact surface which shows the width of the same tapering from a generally constant width posterior portion to a wider portion at the anterior end.

FIG. 1E illustrates a view of another example of a contact surface which shows the width of the same tapering from a generally constant width anterior portion to a wider portion at the posterior end portion.

FIG. 1F illustrates an example of a contact surface that curves to accommodate the curvature in the path taken over the range of motion of the joint.

FIG. 1G illustrates a cross-sectional view of the contact member of FIG. 1F taken along lines 1G-1G.

FIG. 2 illustrates a perspective view of another embodiment of an internal brace 10 for implantation in a patient to treat knee pain.

FIGS. 3A-3B illustrate a perspective view and a sectional view of another embodiment of an internal brace for implantation in a patient to treat knee pain.

FIG. 3C illustrates an alternative embodiment of the compliant member of FIGS. 3A-3B which has transitional compliance.

FIG. 4 illustrates a view of another embodiment of a brace according to the present invention.

FIG. 5 illustrates a bicompartmental system in which a brace of the type described with regard to FIG. 4 above is implanted on the medial side of the knee, and another brace of the type described with regard to FIG. 4 above is implanted on the lateral side of the knee.

FIG. 6 illustrates an internal brace that is attached to the femur and tibia at the knee joint in a manner where portions of the patient's femur and tibia are removed to receive at least the stems of the brace, so that the outer surface of the brace is substantially flush with the bone surfaces of the femur and tibia.

FIG. 7A illustrates another embodiment of an internal brace according to the present invention.

FIGS. 7B-7F schematically illustrate partial views of various embodiments of an axially rigid yet bendable member useable for fixation of one or more brace components described herein.

FIG. 8 illustrates a brace that can be custom configured to provide support during one or more portions of the gait cycle.

FIG. 9 illustrates a brace provided with a sheath according to the present invention.

FIG. 10A illustrates an embodiment of an internal brace in which the bearing surfaces and the tapering portions extend further into the knee joint than embodiments previously shown.

FIG. 10B shows the embodiment of FIG. 10A after components of the arrangement in FIG. 10A have been removed and replaced with the portions shown in FIG. 10B that have much shorter bearing surfaces.

FIGS. 10C-10D show examples of braces in which the dimensions of the bearing surfaces in the anterior-posterior direction have been altered, relative to one another.

FIG. 11A shows an embodiment of a brace that, like previously described embodiments, includes removably attached portions.

FIG. 11B illustrates an anterior view of a portion of the brace of FIG. 11 that has been manufactured as a deformable component that is deformed during the attachment procedure to generally follow and fit to the contours of the bone in the location where it is to be attached.

FIG. 11C illustrates an anterior view of a portion of the brace of FIG. 11 that has been manufactured with a contoured configuration to generally follow and fit to the contours of the bone in the location where it is to be attached.

FIG. 12 illustrates an internal brace implanted on the lateral side of a knee joint for lateral side support, according to the present invention.

FIG. 13 illustrates a bicompartmental system in which an internal brace of the type described with regard to FIG. 12 above is implanted on the lateral side of the knee, and another internal brace of the type described with regard to FIG. 12 above is implanted on the medial side of the knee.

FIG. 14 shows an embodiment of a brace in which the bearing surface of the femoral portion is provided with one or more (preferably a plurality of) ball or roller bearings.

FIG. 15 illustrates an embodiment of an internal brace that is provided with axial length adjustability.

FIG. 16A illustrates an internal brace 10 having been implanted intramedullarly in the femur and tibia.

FIG. 16B illustrates an embodiment of bearing surface configurations for the internal brace of FIG. 16A.

FIG. 17 illustrates another embodiment of an internal brace having been implanted intramedullarly in the femur and tibia.

FIG. 18 illustrates another embodiment of an internal brace that is implanted external of the joint.

FIG. 19A-19C illustrate an embodiment of an internal brace in which relative rotation of the components occurs superiorly of the knee joint, preferably near or at the center of rotation of the knee joint.

FIG. 20A illustrates another embodiment of a brace that can be attached medially or laterally (or one brace attached medially and one brace attached laterally) to the femur and tibia.

FIG. 20B shows a cross sectional partial view of the device of FIG. 20A taken along line 20B-20B.

FIG. 20C illustrates a variant of the embodiment of FIG. 20A, in which the core may be formed as one or more ball bearings, as schematically illustrated in FIG. 20C.

FIG. 20D schematically illustrates that the contact surfaces may be flat in the medial lateral direction and optionally may be provided with edges that deter malalignment of the components.

FIGS. 21A-21B illustrate a variant of the brace of FIG. 20A, which is installed similarly to and functions similarly to the brace of FIG. 20A.

FIG. 22 illustrates a magnetic feature that be incorporated into various embodiments of the braces according to the present invention.

FIG. 23 illustrates one example of a brace according to the present invention where a contact surface has been provided with a cam surface in the anterior posterior direction (right to left in FIG. 23).

FIGS. 24A-24D illustrates an embodiment where the relative amounts of load can be varied over the gait cycle, without the need to move the anchoring locations of the upper and lower portions of a brace according to the present invention.

FIG. 25 illustrates an internal brace according to the present invention in which a compliant feature is provided in one of the portions in the brace.

FIGS. 26A-26B illustrate an embodiment of an internal brace according to the present invention that is configured to be implanted against the medial or lateral side of a knee joint.

FIGS. 27A-27B show a side view and an anterior view, respectively, of a device employing an intra-articular tibial component, according to the present invention.

FIGS. 28A-28B show an anterior view and a side view, respectively, of a single component brace according to the present invention.

FIGS. 29A-29B show an anterior view and a side view, respectively, of a brace configured for treatment of trauma.

FIG. 30 illustrates an internal brace according to the present invention implanted on the lateral side of the knee joint, in combination with an energy manipulation system implanted on the medial side of the knee joint.

FIGS. 31A and 31B show an anterior-posterior view and a lateral view of an internal braced implanted to an ankle joint.

FIG. 31C illustrates a sectional view of a portion of the upper component of the brace of FIG. 31A, taken along line 31C-31C in FIG. 31A.

### DETAILED DESCRIPTION OF THE PREFERRED EMBODIMENTS

Before the present devices and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a bearing” includes a plurality of such bearings and reference to “the screw” includes reference to one or more screws and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

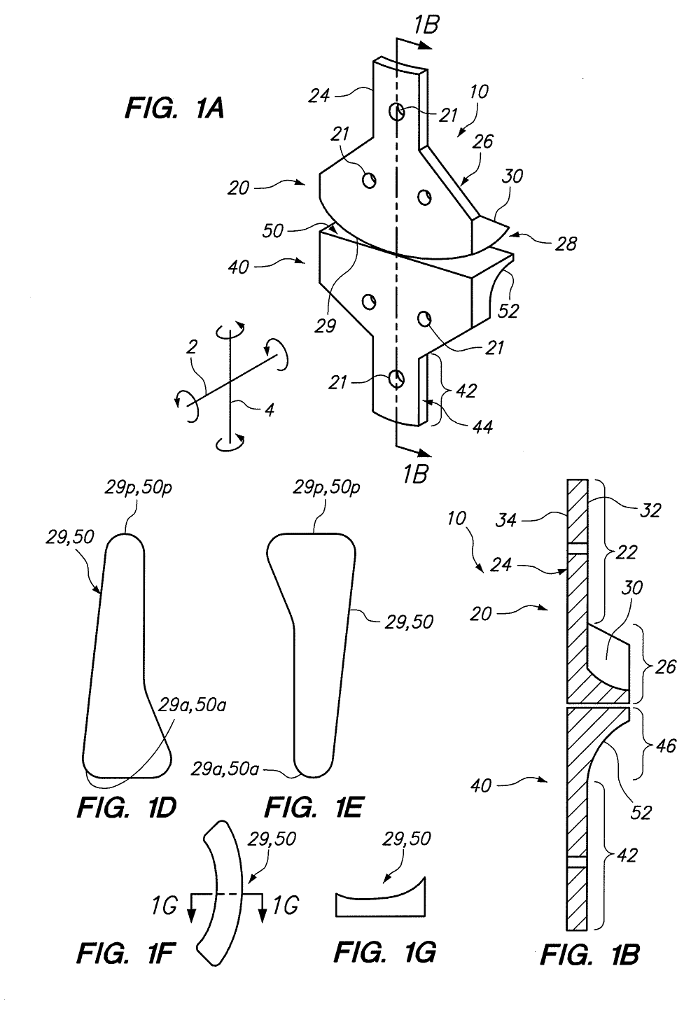

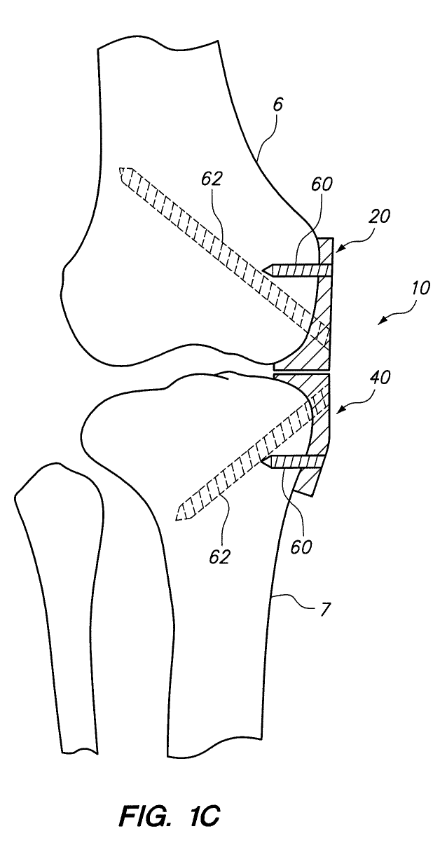

An implantable brace according to the embodiments of the present invention includes at least one component for connection to at least one bone from which a joint is formed. FIGS. 1A-1B illustrate a perspective view and a sectional view of an embodiment of an internal brace 10 for implantation in a patient to treat knee pain. It is noted here that although specific embodiments described herein are adapted to treatment of the knee joint of a patient, that they can also be adapted to treatment of other joints in the body, including, but not limited to: finger joints, toe joints, elbow joints, etc. Internal brace 10 includes a femoral component 20 and a tibial component 40. The femoral component 20 is configured to be attached to a distal end portion of a patient's femur. The femoral component 20 includes an upper portion 22 that includes an elongated stem 24. Femoral component 20 further includes a lower portion 26 tapering from the upper portion 22 outwardly as it extends downwardly, into a condylar protrusion 28 that extends into the space in the joint between the bones. The condylar protrusion 28 has a convex lower surface 29. The upper surface 30 of the condylar protrusion 28 is contoured to generally conform to the condyle of the femur or a portion of the condyle of the femur that has been removed of the patient at the distal end of the femur.

The upper portion 22 comprises an inner surface 32 configured to be attached to the femur and an outer surface 34 that is external of the femur when the inner surface 32 is attached to the femur and the internal brace 10 has been implanted.

The tibial component 40 is configured to be attached to a proximal end portion of a patient's tibia. Tibial component 40 includes a lower portion 42 that includes an elongated stem 44. An upper portion 46 tapers outwardly from the lower portion 42 as it extends upwardly therefrom, to form an upper tray 48 having a flat upper surface 50 for engaging the convex lower surface 29 of the condylar protrusion 28 so as to enable relative rotation between the femoral component 20 and the tibial component 40. The lower surface 52 of the tray 48 is contoured to generally conform to the contour of the tibial plateau or a portion of the tibial plateau that has been removed. By providing surface 50 as a flat surface, not only are components 20 and 40 able to rotate relative to one another about a transverse axis 2, they are also able to rotate about a longitudinal axis 4 relative to one another. Further, components 20 and 40 are also permitted translation relative to one another in at least the anterior-posterior direction. Thus, brace 10 allows This allows relative longitudinal axial rotation of the femur and tibia and anterior-posterior translation during flexion and extension movements of the knee, so that device 10 does not restrict the relative longitudinal axial rotations and anterior-posterior direction translations that naturally occur during flexion and extension in the gait cycle, as it would if surface 50 were replaced by a concave surface conforming to convex surface 29.

To accommodate for the resultant changes in position of the contact surfaces 29 and 50 from the longitudinal axial rotations and anterior-posterior translations during the gait cycle, one or both of contact surfaces (and the underlying or overlying support structure) can be configured to have at least a portion thereof that is substantially wider than another portion thereof. FIG. 1D illustrates a top view of one example of contact surface 50 (or a bottom view of surface 29) which shows the width of the surface tapering from a generally constant width posterior portion 50p, 29p to a wider portion at the anterior end portion 50a, 29a. Thus, anterior portion 29a, 50 is wider than posterior portion 29p,50p. FIG. 1E illustrates a top view of another example of contact surface 50 (or a bottom view of surface 29) which shows the width of the surface tapering from a generally constant width anterior portion 50a, 29a to a wider portion at the posterior end portion 50p, 29p. Thus, posterior portion 29p, 50p is wider than anterior portion 29a,50a. Alternative embodiments may have variations in the amount of taper and the location along the length of the surface where the taper begins. Use of the configuration of FIG. 1D versus that of FIG. 1E may depend upon whether the brace is being implanted on the medial side or the lateral side. The contact surface 39, 50 may be curved to conform to the track that a contact surface of one of the natural bones takes relative to the contact surface of another of the natural bones, for example, the contact surface may be formed with a curvature in the plane that is normal to the line 1B-1B in FIG. 1A, for example, as illustrated in FIG. 1F. In this example, the contact surface is shaped similarly to a meniscus, although other curved shapes may be employed. Further, the contact surface may be curved in other planes, such as a plane normal to that shown in FIG. F, as illustrated by the cross-sectional illustration of FIG. 1G taken along line 1F-1F of FIG. 1F.

FIG. 1C is a sectional view of the internal brace of FIGS. 1A-1B implanted in the medial joint of a knee of a patient. As shown the upper component 20 is configured to conform to the external surface of the patient's femur 6 and the lower component 40 is configured to conform to the external surface of the patient's tibia 7. Additionally or alternatively, portions of the patient's femur 6 and tibia 7 may be removed to receive the stems 24 and 44 so that they are at least partially recessed into the femur 6 and tibia 7 and may even be flush therewith.

The components 20 and 40 are secured by one or more fasteners, such as screws, such as locking screws 60 and bicortical screws 62 passed through openings 21 and screwed into the bone of the femur 6 and tibia 7, respectively. Alternative fasteners include, but are not limited to dynamic lag screws. Further alternatively, one or both of upper and lower stems 24, 44 may be formed as blade plates and attached using any of the fasteners described. Screws passing through the lower portion 26 of the femoral component 20 may be angled upwardly as they are screwed into the femur 6 to avoid critical anatomical landmarks and to achieve better purchase as this portion of the bone is generally stronger. Likewise, the screws passing through the upper portion 46 of the tibial component 40 can be screwed in along a trajectory that is angled downward. Internal brace 10 is implantable underneath the medial collateral ligament (not shown).

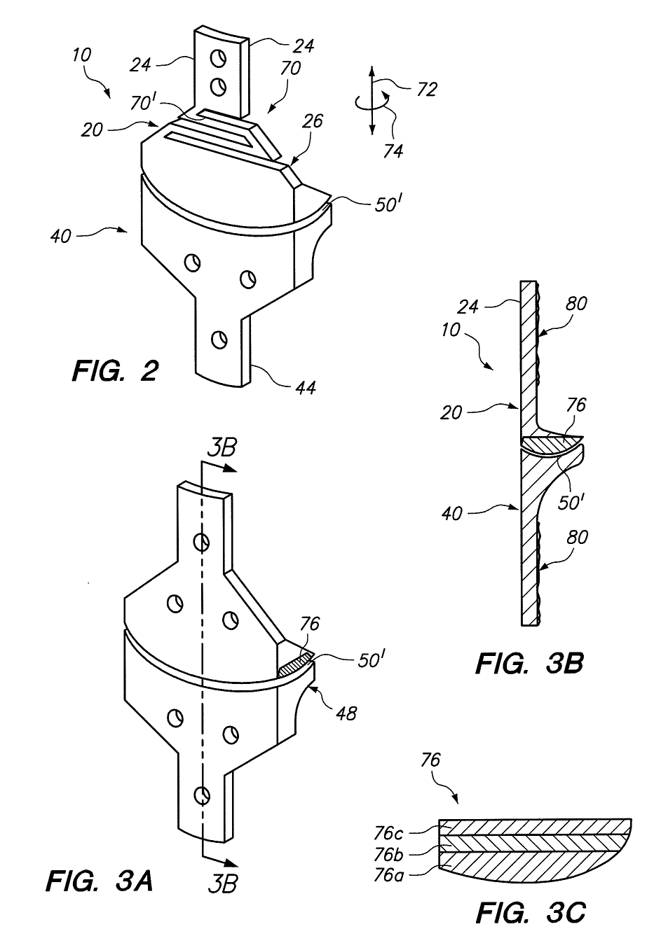

FIG. 2 illustrates a perspective view of another embodiment of an internal brace 10 for implantation in a patient to treat knee pain. This embodiment is similar to that of FIGS. 1A-1C but differs in that it includes compliant member 70 in femoral component 20. Compliant member 70 provides compliance in the internal brace 10, so that the upper portion 24 can move axially relative to the lower portion 26, and thereby the femur and tibia are allowed a limited amount of relative axial movements to one another (i.e., in the directions of arrows 72). Additionally, compliant member 70 acts to allow the space between the bones to close and open thus mimicking the fluid movement and loading/unloading of the cartilage of a healthy articular joint. Compliant member 70 further allows relative rotation between the upper and lower portions 24 and 26, thereby allowing limited relative longitudinal axial rotation of the femur and tibia during flexion and extension movements of the knee, so that device 10 does not restrict the relative longitudinal axial rotations that naturally occur during flexion and extension. Compliant member 70 acts to absorb at least a portion of the load and alter the load carrying and load transfer characteristics of the brace. Struts 70s can be varied (e.g., by altering the thicknesses and/or lengths of struts 70s) to alter the characteristics (e.g., spring constant in axial and or rotational directions) of the compliance provided by compliant member 70.

It is noted that alternative to what is shown in FIG. 2, the compliant member can be provided in the tibial component between the upper and lower portions to achieve the same effects. Further alternatively or additionally, a compliant member can be provided between the femoral and tibial components, e.g., between contact surfaces 29 and 50. It is further noted that compliant member 70 could be incorporated into the embodiment of FIGS. 1A-1C. Likewise, rather than providing the embodiment of FIG. 2 with a concave upper surface 50′ of the upper tray 48, so that the upper surface 50′ conforms to the convex lower surface of upper portion 20, the upper surface of the tray 48 could be provided as a flat surface 50 like that of FIG. 1A. More generally, the features of each embodiment described herein are combinable with those of other embodiments unless it would not be possible to do so, e.g., where one feature is an alternative to another feature and therefore replaces that feature or the substitution or combination would make the embodiment inoperative.

FIGS. 3A-3B illustrate a perspective view and a sectional view of another embodiment of an internal brace 10 for implantation in a patient to treat knee pain. This embodiment is similar to that of FIGS. 1A-1C but differs in that it includes a compliant material 76 lining the convex surface 29. This compliant material may be made of a compliant biocompatible polymer such as an elastomer, and functions as a bearing surface for load absorption. During loading, compliant material 76 compresses. Accordingly, compliant material 76 allows limited relative axial movements between upper portion 24 and lower portion 26, and thereby the femur and tibia are allowed a limited amount of relative axial movements to one another. Additionally, compliant material 76 acts to allow the space between the bones to close and open thus mimicking the fluid movement and loading/unloading of the cartilage of a healthy articular joint. In this regard, compliant material may be modeled to more closely mimic the differences in compliances in the natural materials forming the joint. For example, compliant material may be formed to having varying compliance, with a portion forming the contact surface of the compliant material being most compliant (mimicking the meniscus, for example), an intermediate portion having intermediate compliance (mimicking the transition from meniscus to bone, for example), and a portion that is mounted to the metal member having the relatively least compliance (mimicking the bone further densifying at distances further away from the meniscus, for example). FIG. 3C illustrates an example of compliant member 76 configured to have transitional compliance. The portion 76a of member 76 that is furthest from the interface with the rigid member 20 and includes the contact bearing surface has the most compliance, and the portion 76c that interfaces with the metal upper member 20 has the least compliance of the portions of member 76. Portion 76b has a compliance that is less than that of 76a, but greater than that of 76c. Accordingly, member 76 provides transitional compliance, with the most compliance being provided at the portion containing the contact surface and with the compliance transitionally decreasing in the direction toward the metal component 20. A transitional compliant member is not limited to three portions each having a different compliance, but may include two portions or more than three portions. Further alternatively a transitional compliant member may be formed to have continuously varying compliance in a direction from a location furthest from where it is mounted to the surface that interfaces with the member that it is mounted to. In any of these examples, the transition in compliance will typically transition from least compliance at the end where the transitional compliant member is mounted, to most compliance at or near the contact surface that is furthest away from the surface where the transitional compliance member is mounted. Transition in the compliance be achieved by providing spring members having varying compliance, or other mechanical compliance members, alternative to, or in addition to materials having different compliance characteristics, as in the example of FIG. 3C.

It is noted that alternative to what is shown in FIGS. 3A-3C, compliant material can be provided on the upper surface 50′ (or 50) of the tray 48 to achieve the same effects. It is further noted that compliant member 70 could be incorporated into the embodiment of FIGS. 3A-3B, and/or that a flat surface 50 can be provided alternatively to concave surface 50, as features among different embodiments are combinable, if possible, as noted above.

The bone contacting surfaces of the upper and lower portions 20 and 40 may be configured to enhance osteointegration. Osteointegration enhancers include, but are not limited to, coatings, such as hydroxyapatite or other calcium phosphate compositions, bone morphogenetic proteins, collagens, or other proteins that have been shown to help induce osteointegration or osteogenesis, roughened or porous surfaces, or other treatments known and used in the art to enhance bone growth. FIG. 3B shows osteointegration enhancers 80 provided on the bone contacting surfaces of the upper stem 24 and lower stem 44. However, osteointegration enhancers as described above may be provided on any surface of a device described herein in which it is desired to encourage bone attachment thereto.

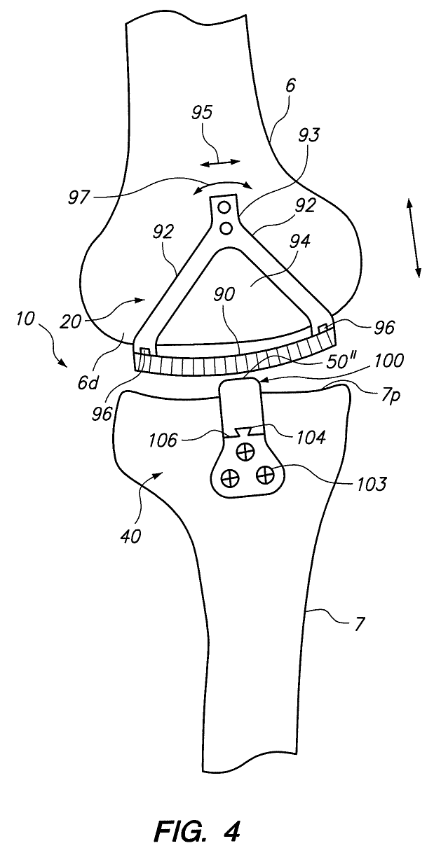

FIG. 4 illustrates a view of another embodiment of a brace 10′ having been implanted by attachment to the femur 6 and tibia 7, respectively. In this embodiment, the upper portion 20 that is attached to the femur 6 includes a suspended compliant member 90 that functions as both a bearing surface and a compliant member in use. As shown, upper member 20 is formed in a triangular configuration, wherein two sides of the triangular member are formed by struts 92 and the third side is the suspended compliant member 90. The upper portion is fixed to the femur 6, along a portion 93 that is opposite to suspended compliant member 90, using screws, and optionally osteointegration enhancer 80, such as by any of the manners described above. The triangular configuration is used here as it is known to provide excellent structural rigidity. However, other configurations may be alternatively used, in which one or more struts 92 connects a suspended compliant member 90 to the femur 6 so as to function as described hereafter.

Suspended compliant member 90 is flexible, so that it functions to flex under loading when contacting the upper surface 50″ of the lower, tibial component 40. The suspended compliant member 90 extends distally of the distal end 6d of the femur 6 when attached to the femur as shown in FIG. 4. A space or suspension distance 94 exists between the suspended compliant member 90 and fixed portion 93. Under walking or running loads, the suspended compliant member 90 deflects somewhat toward the femur, thereby changing the radius of curvature somewhat of at least the deflected portion of the suspended compliant member 90, but not changing it sufficiently to interfere with sliding motions against the opposing bearing element. In this way, suspended compliant member 90 functions as a bearing surface and acts to allow the space between the bones to close and open thus mimicking the fluid movement and loading/unloading of the cartilage of a healthy articular joint. The extent of deflection of compliant member 90 determines the extent of femur/tibia contact in the functional region of the device (where the load is being carried). As compliant member 90 deflects, the femur and tibia come closer together and carry increased loads. The increased loads carried by the femur and tibia thus increase as the amount of deflection of compliant member 90 increases. Compliant member 90 thus provides compliance in the brace 10′, so that relative axial motion between the upper portion 20 and lower portion 40 can occur. This in turn allows relative axial movement between the femur 6 and tibia 7.

One or both components 20, 40 may be adjusted in the axial direction indicated by the relatively vertical arrows in FIG. 4. These adjustments cause a relative variation in the amount of loading of the brace. Also, in the case of a compliant brace 10, such as the one shown in FIG. 4, for example, this type of adjustment alters the amount of absorption provided by the compliant member(s) down to a minimum amount above which distraction occurs.

In the use of a non-compliant brace 10, the adjustment of the brace in the axial direction alters the amount of distraction of the joint by the brace. These adjustments can be made by altering the locations on the femur 6 and tibia 7 that the upper and lower components are screwed into. Alternatively, one or more adjustment mechanisms may be provided in the brace 10 so that the anchoring locations to the femur 6 and tibia 7 do not need to be changed, but the alteration can be made by altering the adjustment mechanism. One such adjustment mechanism is illustrated in FIG. 15, for example.

Suspended compliant member 90 is removably fixed to the one or more struts 94. Thus, suspended compliant member 90 can be removed and replaced, as needed, either with a suspended compliant member having the same specifications as the one being replaced, or with a suspended compliant member having a different curvature and/or different elastic bending modulus than the one being replaced. Removable fixation of the suspended compliant member to the one or more struts may be by screws 96, which may be countersunk so as not to interfere with the bearing function of member 90.

Lower portion 40 is fixed to the tibia 7 when brace 10′ is implanted, as shown in FIG. 4. A fixed base portion 103 is screwed (and optionally, osteointegration enhancers 80 may be used) to fix base portion 103 to the bone of the tibia 7. Opposing bearing member 100 opposes suspended compliant member 90 and is removably attached to base portion 103. Opposing bearing member 100 extends proximally of the proximal end 7p of the tibia 7 when lower portion 40 is attached to the tibia as shown in FIG. 4. Under walking or running loads, the opposing bearing member 100 does not deflect as it rides against the suspended compliant member 90 and slides relative thereto. Further, since the surface 50″ of opposing bearing member 100 is flat or slightly convex, relative rotation between bearing 100 and suspended compliant member 90 is also permitted. Accordingly, this allows relative rotation between the upper (femoral) component 20 and the lower (tibial) component 40. This allows relative longitudinal axial rotation of the femur and tibia during flexion and extension movements of the knee, so that device 10 does not restrict the relative longitudinal axial rotations that naturally occur during flexion and extension during the gait cycle.

Opposing bearing member 100 is made of a relatively rigid material, such as a biocompatible metal, alloy, or hard, thermosetting polymer. Opposing bearing member 100 is removably attached to base 103 by a fixation arrangement including, but not limited to a dovetail joint 104 and/or one or more set screws 106. Additionally or alternatively, portions of the patient's femur 6 and tibia 7 may be removed to receive the bases 93, 103 and portion of struts 92 (and optionally, bearing 100) so that they are at least partially recessed into the femur 6 and tibia 7 and may even be flush therewith.

The placement/location in which fixed base portion 93 is fixed to the femur 6 may vary, both in an anterior/posterior direction (arrows 95) as well as angularly relative to the longitudinal axis of the femur 6 (arrows 97) to adjust the brace according to whether all or only part of the gait cycle of the knee joint is to be supported. For example, by rotating the upper portion 90 clockwise and translating the fixed base portion to the left in FIG. 4, relative to the femur 6, while leaving the lower portion 40 fixed in the location shown, brace 10 can be configured to not support the knee joint in full extension (configuration shown), but to support during at least a portion of the gait cycle in which the knee is in partial and/or full flexion. Conversely, the relative location of the upper portion can be fixed to treat the joint only in full extension. Further relative fixation locations can be used to customize the amount of the gait cycle during which the knee joint is supported, as well as the relative amount of support provided in various portions of (or all) of the gait cycle that is supported.

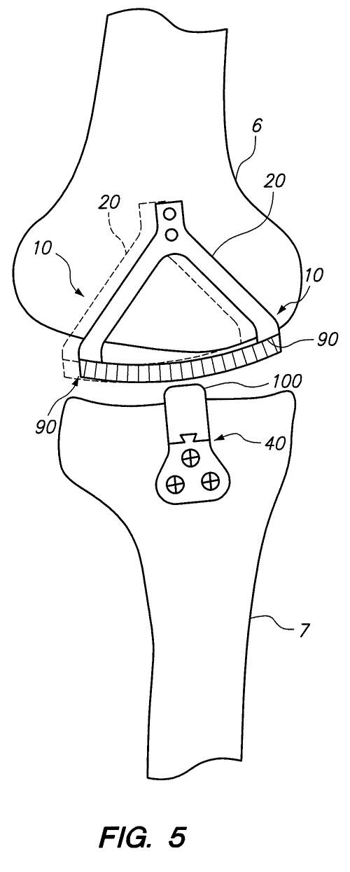

The brace 10′ of FIG. 4, like all other braces described herein, can be implanted on either the medial side of the knee or the lateral side of the knee, on the left knee or the right knee. Further, braces described herein can be implanted as a pair, one on the medial side of the knee and one on the lateral side of the knee. FIG. 5 illustrates a bicompartmental system in which a brace 10′ of the type described with regard to FIG. 4 above is implanted on the medial side of the knee, and another brace 10′ of the type described with regard to FIG. 4 above is implanted on the lateral side of the knee. Because the pathway defined by the contact between the bearing surfaces of the femur 6 and tibia 7 is not the same on the medial side as it is on the lateral side, the upper portion 20 (phantom lines) of the brace 10′ on the lateral side is not placed directly opposite the placement of the upper portion 20 (solid lines) of the brace 10′ on the medial side, to account for the different pathways along the medial compartment compared to the lateral compartment during the normal gait cycle, from extension to flexion back to extension again. The translation of the femur relative to the tibia on the lateral side is greater than the translation on the medial side. This results in a complex motion of the knee, including relative axial rotation between the femur 6 and tibia 7, and different contact pathways along which the bearing surfaces of the devices 10 interact. The rotation of the knee is not along a central pivot axis, but is much more complex, with the medial and lateral sides experiencing different amounts of lateral sliding during relative rotation between the femur 6 and the tibia 7. The braces of the present invention can be placed to account for these differences when a pair of braces is installed, one on the medial side of the knee and the other on the lateral side of the knee. Accordingly, the axis of rotation of the upper portion 20 of the brace 10′ on the lateral side of the knee in FIG. 5 may be is offset in the anterior-posterior direction relative to the axis of rotation of the upper portion 20 of the brace 10′ on the medial side of the knee to accommodate the different paths taken during the gait cycle. The lower portions 40 are in alignment in FIG. 5 so that the lower portion 20 of the lateral brace is not visible in FIG. 5.

The differing paths of the medial and lateral compartments may be accommodated by the same type of brace 10 placed at relatively different opposing positions on the medial and lateral sides of the knee. Alternatively, different types of devices 10 may be used on the medial and lateral sides of the knee respectively, wherein the different braces 10 are designed to accommodate the different paths required for the two sides. In this case, such braces 10 may be implanted in directly opposing positions on the medial and lateral sides of the knee and still accommodate the differing paths of motion on the respective medial and lateral sides. Further alternatively, different types of braces 10 can be implanted at relatively different opposing positions on the medial and lateral sides to accommodate the different path requirements.

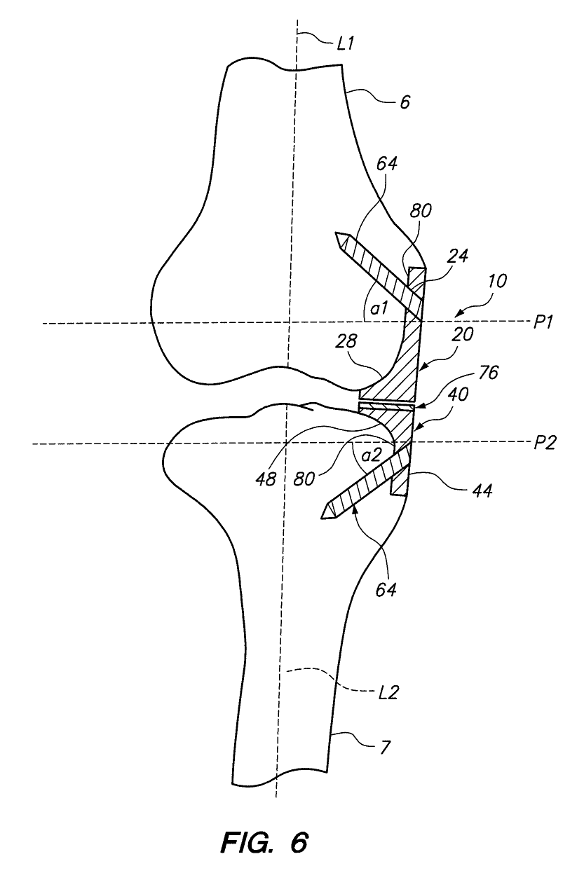

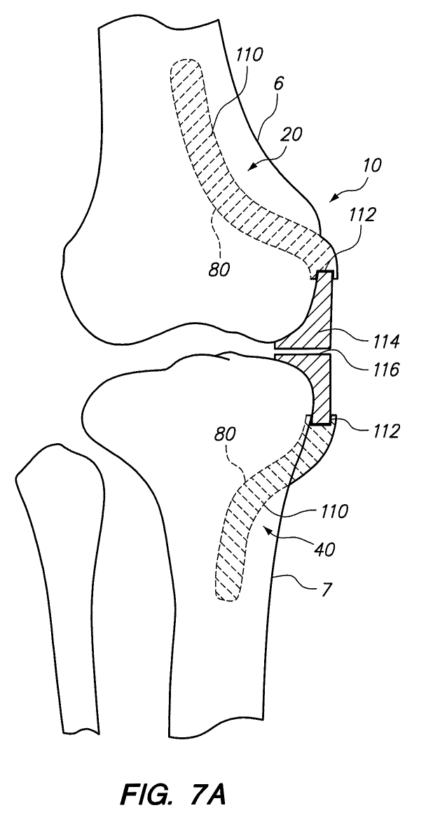

FIG. 6 illustrates an internal brace 10 that is attached to the femur 6 and tibia 7 at the knee joint in a manner where portions of the patient's femur 6 and tibia 7 are removed to receive at least the stems 24 and 44, so that the outer surface of the internal brace is substantially flush with the bone surfaces of the femur 6 and tibia 7, as shown in FIG. 6. Optionally, portions of the condyles and/or cartilage on the femur 6 and tibia 7 may be removed to receive at least portions of the protrusions 28, 48 for greater stability and/or to remove damaged or diseased bone. Further, removal of at least a portion of one or both of the protrusions 28, 48 may be performed to maintain natural alignment of the knee so that an additional thickness is not added by overlaying those features with the brace 10 components. Bearing surface 76 is placed on the upper surface of the lower (tibial) component 40 as shown, but alternatively may be placed at the bottom bearing surface of the femoral (upper component) 20. Bearing surface 76 comprises a compliant material, which may be made of a compliant biocompatible polymer such as an elastomer, and functions as a bearing surface and acts to allow the space between the bones to close and open thus mimicking the fluid movement and loading/unloading of the cartilage of a healthy articular joint. During loading, compliant material 76 compresses. Accordingly, compliant material 76 allows limited relative axial movements between upper portion 20 and lower portion 40, even after the bearing surfaces make contact.

The bases of the upper and lower portions 20 and 40 in this case are anchored to the femur 6 and tibia 7, respectively using compression screws 64. The compression screw(s) 64 attaching the upper portion 20 to the femur 6 may be driven into the femur in an angularly upward direction, such that the compression screw(s) 64 points away from the upper portion 20 in an angularly upward direction, angling upwardly from a horizontal line P1 that is perpendicular to the longitudinal axis L1 of the femur 6. The compression screw(s) 64 attaching the lower portion 40 to the tibia 7 may be driven into the tibia in an angularly downward direction, such that the compression screw(s) 64 points away from the upper portion 20 in an angularly upward direction, angling upwardly from a horizontal line P2 that is perpendicular to the longitudinal axis L2 of the tibia 7.

By insetting internal brace 10 at least partially into the bones 6, 7 such that the internal brace 10 is flush with the bone surfaces, or at least extends from the surfaces less than a brace that is simply attached to the outer surfaces of the bones 6 and 7, this causes the internal brace 10 to be less of an obstruction to the medial ligament. Consequently, internal brace 10 is more easily implanted under the medial ligament without causing complications to the medial ligament. Additionally, relative motions of the internal brace component are less likely to irritate or otherwise cause problems with the medial ligament or other soft tissue structures. Thus, this results in a lower profile implant, causing less skin irritation and less irritation to other soft tissues.

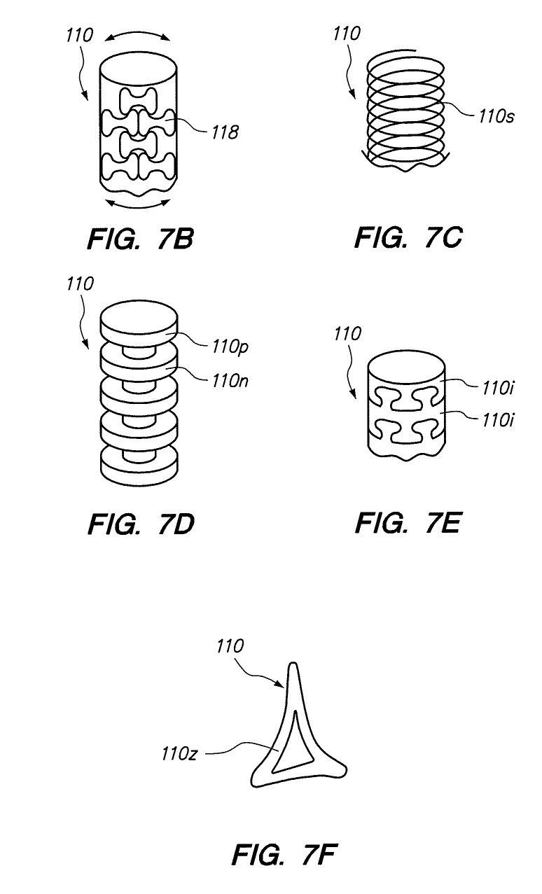

FIG. 7A illustrates another embodiment of an internal brace 10 according to the present invention. In this embodiment, the majorities of the upper and lower portions 20 and 40 are implanted into the femur 6 and tibia 7, respectively. Thus, only a small proximal end portion of each of the internally implanted members 110 of the upper and lower members 20, 40 are external of the bones 6, 7. Members 110 are like intramedullary nails or other axially incompressible, but flexible (bendable) members 110 that provide column strength due to their axial incompressibility, but allow the members to follow the contours of the better structurally supporting bone of the femur 6 and tibia 7 that they are implanted into. The exposed proximal end portions include sockets, or other connection features 112 that allow removable bearing components 114 and 116 to be removably attached thereto. Components 114, 116 are rigid and generally follow the contours of the condyles and cartilage to which they are being fitted. Optionally, at least a portion of the cartilage and/or condyle of the femur 6 and/or the tibia 7 may be removed to allow a respective bearing component 114, 116 to be received into a cut out recess. The bearing surfaces of the components 114, 116 may be incompressible (e.g., metal), or, alternatively, at least one of these surfaces may be compliant to allow some axial movement. Members 110 will typically be driven into the respective bones 6 and 7 after boring an entrance hole through the cortical bone. By driving the member 110 in, a compression fit is formed, and, with healing, bone grows into the members 110 which are typically provided with some form of osteointegration enhancement features 80.

FIGS. 7B-7F schematically illustrate partial views of various embodiments of axially rigid yet bendable member 110 that can be used in the embodiment of FIG. 7A. In FIG. 7B, member 110 is a metallic tube (e.g., stainless steel, titanium, titanium alloy or the like) that has cutouts 118 formed therein so that the remaining metal forms a series of interconnecting I-beam shapes along the axial direction, thus rendering the tube relatively axially incompressible. However, the cutouts 118 allow bending in the directions of the arrows.

In FIG. 7C, member 110 comprises an incompressible spring 110s that is axially incompressible, but flexible (bendable), thereby providing column strength due to the axial incompressibility, but allowing member 110 to bend to follow the contours of the better structurally supporting bone of the femur 6 and tibia 7 that they are implanted into.

In FIG. 7D, member 110 comprises a profiled or notched rod 110r that is axially incompressible, wherein notches 10n allow some bending to take place, such that member 110 provides column strength due to the axial incompressibility, but bends to follow the contours of the better structurally supporting bone of the bone that it is implanted into.

In FIG. 7E, member 110 comprises an interlocked ring assembly comprising a plurality of interlocked rings 110i that form a column or cylinder that is axially incompressible, but flexible (bendable), thereby providing column strength due to the axial incompressibility, but allowing member 110 to bend to follow the contours of the better structurally supporting bone of the bone that it is implanted into.

In FIG. 7F, member 110 comprises a Zickle rod 110z that is axially incompressible, but flexible (bendable), thereby providing column strength due to the axial incompressibility, but allowing member 110 to bend to follow the contours of the better structurally supporting bone of the bone into which it is implanted.

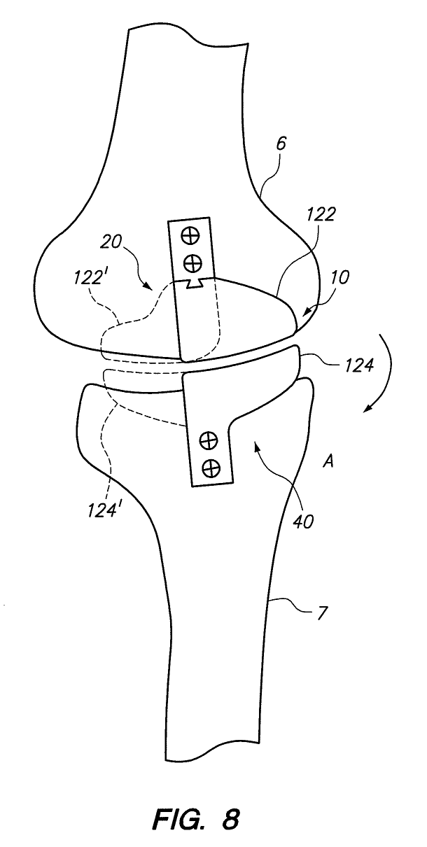

FIG. 8 illustrates a brace that can be custom configured to provide support during one or more portions of the gait cycle. As shown, upper bearing portion 122 is configured to make contact with and slide (and, optionally to allow rotation) relative to lower bearing portion 124 when the knee joint is in extension, as shown. During the gait cycle, as the knee bends and the tibia 7 rotates relatively clockwise to the tibia 6 in FIG. 8 as shown (the anterior portion of the knee joint being to the right side in FIG. 8), the bearing surfaces of portions 122 and 124 slide relative to one another until, flexion has occurred to a significant extent that the bearing surfaces of portions 122 and 124 can no longer make contact with one another as they are no longer in alignment. Thus, during the latter part of the flexion phase of the gait cycle, brace 10′, as configured in FIG. 8 does not distract the knee joint, as the upper and lower components 20, 40 do not make contact with one another during that portion of the gait cycle.

Upper bearing portion 122 is removably attached to the upper base portion 126 (which is fixed to bone 6, using screws and optionally, one or more osteoinduction enhancing agents) by a fixation arrangement including, but not limited to a dovetail joint 104 and/or one or more set screws 106. In this way, upper bearing portion can be removed and replaced not only to address a mechanical problem with an existing upper bearing portion 122 by replacing it with an upper bearing portion of the same design, but alternatively, another bearing portion 122′ (shown in phantom) may be put in to cause the brace 10′ to support the knee joint over a different portion of the gait cycle. For example, the portion 122′ shown would distract more towards the flexion portion of the gait cycle and would not support the knee when in the extension configuration shown in FIG. 8. Further alternatively, the bearing portion 124 of lower portion 40 may be configured differently, such as to extend posteriorly (shown in phantom lines) rather than anteriorly as shown in FIG. 8. The decision whether or not to use 124 or 124′ may be impacted, at least in part, by the condition of the cartilage covering those portions of the condyle of the tibia that 124 and 124′ would overlie, where it may be preferable to overlie the more damaged portion (or remove it and replace it with 124 or 124′). Alternatively, brace 10 may be used as a temporary or periodic therapy whereby distraction may be applied and removed without continued disruption of the bone or bone contacting components, as bearing portion 122 need simply be removed, replaced or exchanged. Further optionally, the lower bearing portion may be a full bearing surface, wherein the portion takes up the area shown by both 124 and 124′.

As noted previously, brace 10 may be used to provide temporary full distraction of a joint. For example, bearing portions 122 and 124 may be configured to distract bones over the full extent of the range of motion so that the natural bearing surfaces of the bones, normally contact one another over the range of motion do not contact at all, but are allowed to heal without having to bear any loads. After the temporary period has expired, bearing surface 122 can be exchanged with a differently configured bearing surface designed to allow at least a partial load to the natural bearing surfaces over at least a portion of the range of motion. Further alternatively, bearing portions 122 and/or 124, or the entire brace 10 may be removed after expiration of the temporary period. The temporary period can vary, depending upon the extent and type of damage to the natural bearing surfaces, the characteristics of the individual patient, etc. In one example the temporary period is about three months. In another example the temporary period is about three to six months. However, this method is not limited to any particular temporary period, as it can be carried out for any temporary length of time, and will generally be governed by an approximate time required to provide optimal healing of the natural contact/bearing tissues.

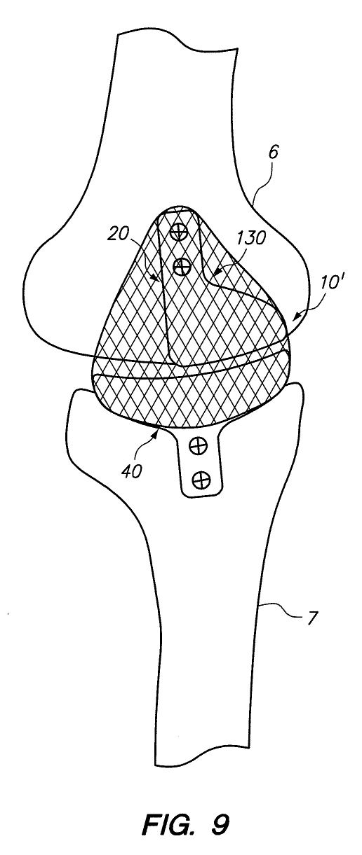

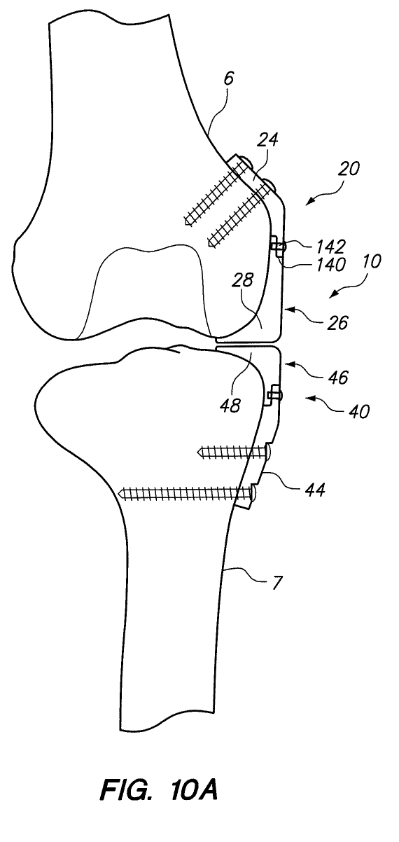

FIG. 9 illustrates a brace 10′ provided with a sheath 130 that encapsulates at least the contact surfaces of the portions that contact one another and perform as bearing surfaces. In the example shown, brace 10′ is of the type shown in FIG. 8, but any other embodiment described herein can be similarly provided with sheath 130. After components 20 and 40 are fixed to the bones 6 and 7, respectively, sheath 130 is fixed to the brace 10′ to cover at least the bearing surfaces (note that the entire upper portion is covered by sheath 130 in the example shown in FIG. 9). Sheath 130 provides a smooth surface that interfaces with the medial ligament and other soft tissues, thereby greatly reducing risks of the medial ligament and other soft tissues being damaged by rubbing on one of the components 20, 40, particularly during movements of one relative to the other. Over time, sheath 130 may become encapsulated by natural tissues as a result of the healing response of the body into which brace 10/sheath 130 are implanted. Optionally, sheath 130 may be formed of a bioresorbable material, such as polylactic acid polymer, polyglycolic acid polymer, copolymers of the same or other biocompatible, bioresorbable materials from which it is possible to construct a sheath. In at least one embodiment, at least the portions of brace 10′ that underlie the medial ligament in any phase of the gait cycle, are covered by sheath 130 to provide a smoother interface with the medial ligament. Further alternatively, sheath 130 may be preinstalled to completely encapsulate at least the bearing surfaces of the brace 10′, prior to fixing components 20 and 40 to the bone. In this case, if the screw holes of one or both components 20, 40 are covered by sheath 130, screws would be driven through the sheath 130 during attachment of the components 20, 40 to the bones 6, 7. Further alternatively, sheath 130 may only encapsulate the condylar portions of the upper and lower components 20, 40 and not the stem portions, so that screws do not need to be driven through the sheath 130 during installation. Sheath 130 may be designed to capture and isolate any wear particles generated from bearing surfaces of the brace 10. Sheath 130 may be snapped or screwed onto the components 20, 40, and/or fixed by other mechanical and/or adhesive means. Sheath 130 may comprise polytetrafluoroethylene or expanded polytetrafluoroethylene to provide a lubricious surface for contact with the medial ligament. Other options include silicone, polyethylene, nylon and/or combinations of these, with or without polytetrafluoroethylene, expanded polytetrafluoroethylene, or other biocompatible lubricious material.

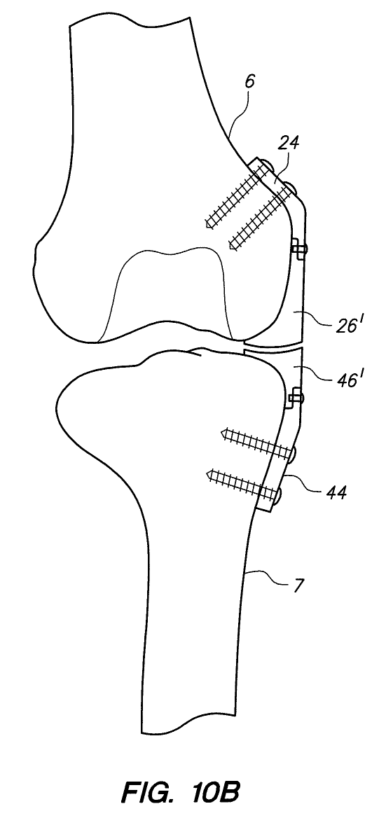

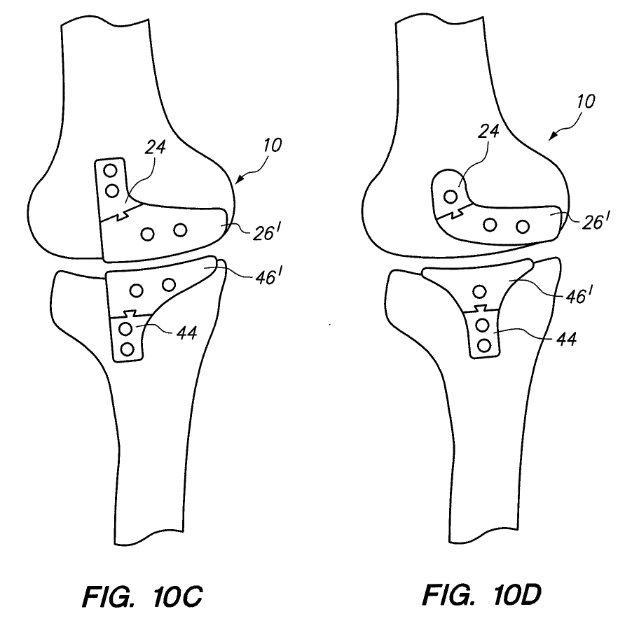

FIG. 10A illustrates an embodiment of an internal brace 10 in which the bearing surfaces and the tapering portions 26, 46 extend further into the knee joint than embodiments previously shown. That is, the condylar portions 28, 48 do not merely form a wedge between the condyles of the femur 6 and tibia 7 to distract the bones 6 and 7 away from one another, but the condylar portions 28,48 in FIG. 10A actually extend into the joint between the condyles of the femur 6 and tibia 7 to cover at least a quarter of the width of the cartilage covering the bone on the medial side (or lateral side, depending upon which side the brace 10 is installed on). Alternatively, as noted above, the cartilage can be removed before overlaying the condylar portion 46 and/or 26. These condylar portions 26, 46 may extend up to about half the width of the cartilage on one side of the knee joint, or up to two thirds, three quarters, or even the entire width of the cartilage on one side. The condylar portions 28, 48 include bearing surfaces that interact with one another in any of the ways already described above.

The tapering portions 26, 46, which include the condylar portions 28, 48 are removably attached to the anchored portions 24, 44 of the upper and lower portions 20, 40. For example, each portion 26, 46 may be fixed to respective portion 24, 44 via a lap joint 140 and screw 142 or other mechanical fixation that can lock the components together, but can be reversed to allow removal and replacement of the component 26, 46. In this way, one or both components 26, 46 can be replaced by like components for correcting a mechanical defect or the like. Alternatively, the components 26, 46 can be replaced by components 26, 46 that have relatively shorter or longer bearing surfaces to alter the distance that they extend into the knee joint. Fixed portions 24 and 44 may be fixed to the femur 6 and tibia 7 respectively, by any of the fixation members and techniques already described above, including, but not limited to use of locking screws, compression screws, bicortical screws and/or osteointegration features.

FIG. 10B shows the embodiment of FIG. 10A after the components 26, 46 of the arrangement in FIG. 10A have been removed and replaced with the portions 26′, 46′ shown in FIG. 10B that have much shorter bearing surfaces, so that they do not extend into the knee joint at locations covering the cartilage, but do form a wedge between the femur 6 and tibia 7 to distract them like in the manner shown and described with regard to previous embodiments.

In addition or alternative to altering the dimensions of the bearing surfaces in the medial-lateral direction as exemplified by what is shown in FIGS. 10A-10B, the dimensions of the bearing surfaces in the anterior-posterior direction can be altered, as illustrated in FIGS. 10C-10D. FIG. 10C illustrates a side view of brace 10 installed on a knee joint, where component 26′ extends fully posteriorly over the femoral condyle, but only a slight distance anteriorly of the longitudinal axis of the femur. Likewise, component 26′ extends posteriorly such that it's bearing surface extends nearly to the posterior end of the tibial condyle, while component 26′ extends only slight anteriorly of the longitudinal axis of the tibia. In FIG. 10D, component 46′ is about symmetrical in it posterior and anterior extent beyond the longitudinal axis of the tibia, while component 26 is provided only over a posterior end portion of the femoral condyle. In this arrangement contact between the bearing surfaces of components 26′ and 46′ occurs only toward the end of the flexion component of the gait cycle. In other portions of the gait cycle (including extension, as shown) the contact surface of component 46′ contacts the natural cartilage of the femoral condyle, as shown in FIG. 10D, if component 46′ extends into the joint space.

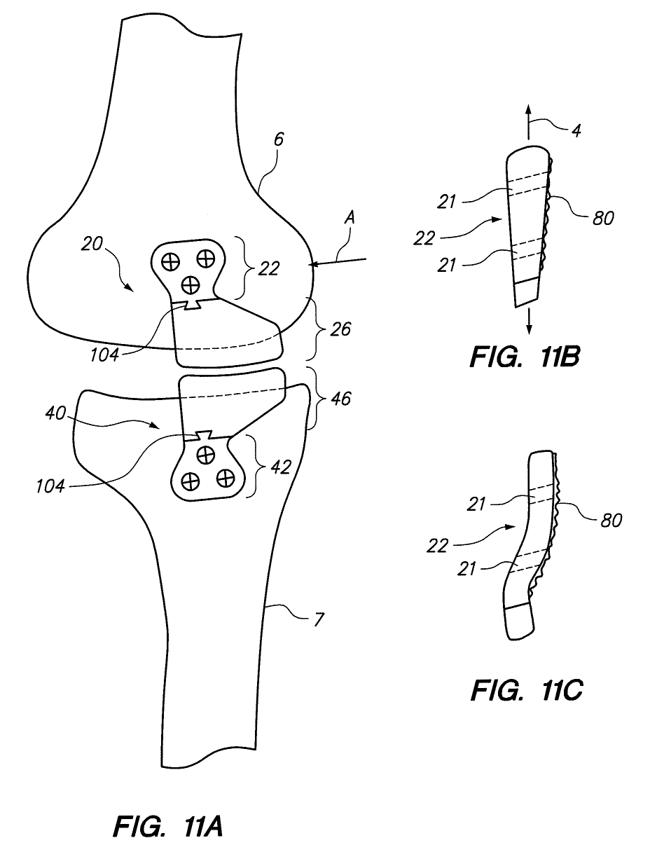

FIG. 11A shows an embodiment of brace 10′ that, like previously described embodiments, includes removably attached portions 26 and 46, so that one or both of these portions can be replaced to remove one or more damaged portions and thereby repair the device 10′, or, alternatively, one or both of portions 26, 46 can be replaced by portions 26, 46 of different design configured to change the support by the brace over one or more portions of the gait cycle.

The base portions (i.e., upper portion 22 of the femoral component 20 and lower portion 42 of the tibial component 40) are fixed to the femur 6 and tibia 7 respectively, and are typically not removed and exchanged when one or both of portions 26 and 46 are replaced. The base portions 22 and 42 may be contoured to follow the contours of the bone of the femur 6 and tibia 7 against which they are anchored. FIG. 11C illustrates an anterior view (i.e., viewing from the direction of arrow A in FIG. 11A) of the portion 22 that is manufactured with a contoured configuration to generally follow and fit to the contours of the bone 6 in the location where it is shown attached to the bone 6 in FIG. 11A. This same method can be applied to portion 42, although it will typically have a different contour designed to generally follow and fit to the contours of the bone 7 in the location where it is shown attached to the bone 7 in FIG. 11A, as the contour of the tibia 7 is generally not the same as the contour of the femur 6.

Alternatively, one or both of portions 22 and 42 can be formed with any surface contour (typically a generally flat or planar surface contour like in FIG. 11B, since this is the most expedient to manufacture and is also a good starting conformation form which to deform the portion to fit the contour of the bone that it is being anchored to) and have mechanical characteristics that render it generally rigid, particularly along the inferior-superior axis 4, and is generally strong overall. However, when using a bending tool or when the portion 22 or 46 is being screwed to the femur 6 or tibia, respectively, the compression and bending forces applied can by the screws deform the portion 22 or 42 to generally follow the contours of the bone that it is being anchored to. Accordingly, in the case of portion 22, the act of anchoring portion 22 to the femur 6 by torquing screws down against the portion 22 through openings 21 and into the bone 6 causes the portion to deform generally to a shape like that shown in FIG. 11C. Regardless of whether portions 22, 42 are rigid or deformable, they may be provided with osteointegration encouraging feature 80 as shown, to encourage bone ingrowth into these portions where they contact the respective bones.

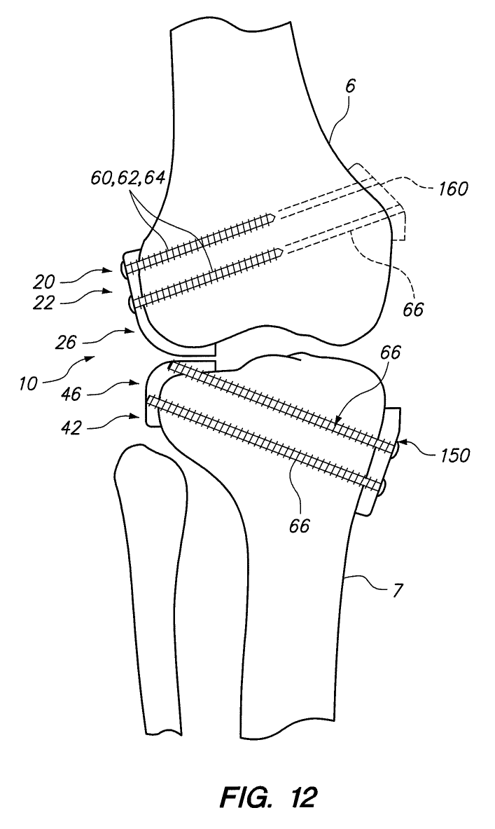

FIG. 12 illustrates an internal brace implanted on the lateral side of a knee joint for lateral side support. In this configuration, the upper portion 22 of the femoral component is fixed to the femur 6 on the lateral side, using locking screws 60, compression screws 64 and/or bicortical screws 62 in any of the manners described above. One or more osteointegration factors/coatings may also be used in a manner as described above. In one embodiment, the tibial component is anchored to the tibia by passing bolts, rods, nails, screws or studs 66 therethrough and connecting them with a second tibial base 150 that is thereby anchored to the medial side of the tibia 7. The medial side base 150 may be provided as a rigid base that is pre-contoured, or may be deformed to follow the contours of the tibial bone on the medial side as the bolt, studs, nails, screws or rods 66 are used to draw the bases 150 and 42 towards one another so as to apply compression to the bone 7. Likewise, the base portions 22 and 42 may be rigid and preconfigured with a contour, or may be deformable in the manner described above with regard to FIGS. 11A-11C.

Optionally, a medial side base 160 (shown in phantom in FIG. 12) may be employed to anchor the femoral component 20.

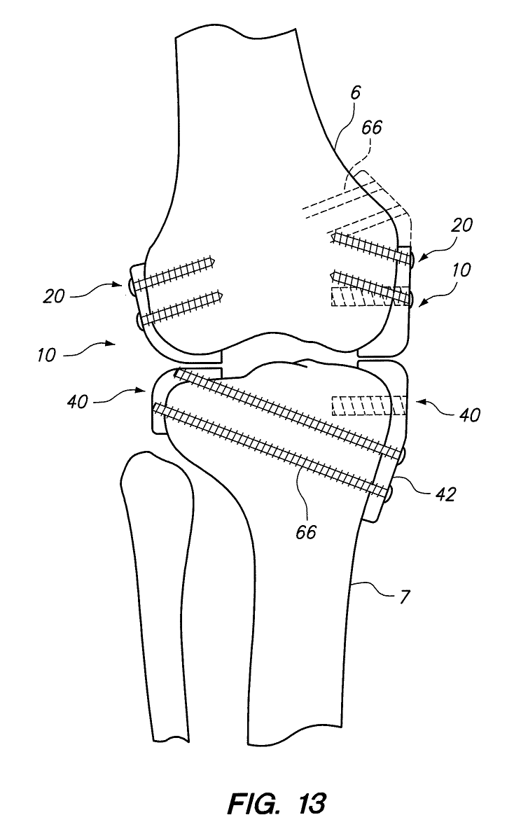

FIG. 13 illustrates a bicompartmental system in which an internal brace 10 of the type described with regard to FIG. 12 above is implanted on the lateral side of the knee, and another internal brace 10 of the type described with regard to FIG. 12 above is implanted on the medial side of the knee. As in FIG. 12, the tibial component 40 of the brace 10 on the lateral side is anchored to a medial side base, which, in this instance, is the base portion 42 of the tibial component 40 of the medial brace 10. Optionally, a compression screw 64 or locking screw 60 may additionally be used to anchor the medial side tibial component 40 to provide additional support for the medial side bearing surfaces. Both femoral components 20 may be anchored in the manner described with regard to FIG. 12. Alternatively, the upper portion 22 of the medial side femoral component may be extended superiorly to be joined by bolts, nails, screws, studs or rods 66 extending through the femur 6 and connected to the lateral side femoral component 20.

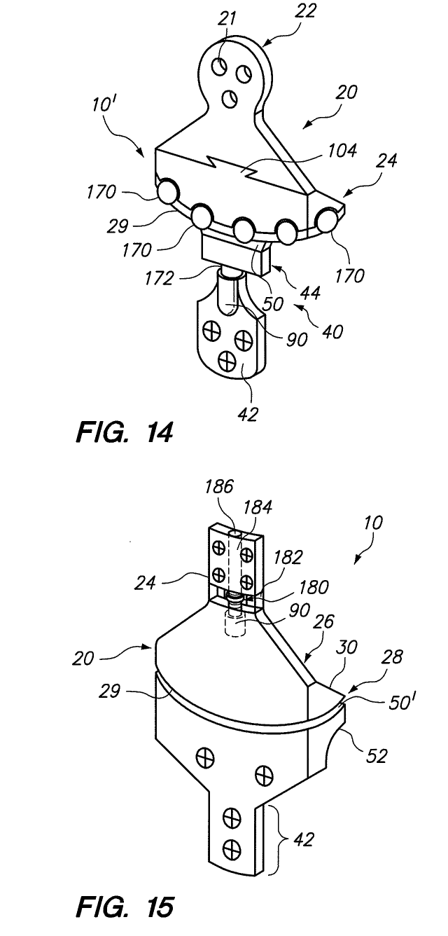

FIG. 14 shows an embodiment of a brace 10′ in which the bearing surface 29 of the femoral portion 20 is provided with one or more (preferably a plurality of) ball or roller bearings 170. Alternatively, the opposing bearing surface 50 of the tibial component 40 could be provided with one or more ball or roller bearings 170. Additionally, the tibial component may be provided with a rotational bearing 172 to allow relative axial rotation between the femur 6 and tibia 7 during the gait cycle as described above. Further optionally, a compliant member and/or dampener 90 may be provided either inferiorly of surface 50 or superiorly of surface 29 (or both) to provide compliance in the brace 10, so that relative axial motion between the upper portion 20 and lower portion 40 can occur and act to allow the space between the bones to close and open thus mimicking the fluid movement and loading/unloading of the cartilage of a healthy articular joint. It also allows relative axial movement between the femur 6 and tibia 7 when brace 10′ has been installed to support the knee joint.

FIG. 15 illustrates an embodiment of an internal brace that is provided with axial length adjustability. A nut 180 is received within the lower portion 26 of the femoral component in a manner such that it is prevented from rotating. Stem portion 24 is telescopically received in a channel 182 formed in lower portion 26 and joined thereto by a threaded connection between screw 184, which passes through stem 24, and nut 180. Screw 184 is prevented from backing out of stem portion 24 or advancing into stem 24 by a pair of shoulders 186, one above the head of the screw 184 and one just below the head of the screw, adjacent thereto. The distance by which stem portion 124 extends from lower portion 26 can be adjusted by rotating the screw 184. Since nut 180 does not turn when screw 184 is rotated, rotation of screw 184 in one direction drives the stem portion 24 into lower portion 26 and thereby shortens the distance by which stem portion extends, and rotation of screw 184 in the opposite direction draws the stem portion 24 out of the lower portion, thereby lengthening the distance by which stem portion 24 extends. Increasing the length by which stem 24 extends out of portion 26, when brace 10 is internally implanted to the knee joint, increases the amount of distraction between the femur 6 and the tibia. Conversely, shortening the length of the stem 24 that extends out of portion 26 decreases the amount of distraction between femur 6 and tibia 7. Alternatively, the adjustment mechanism 180, 182, 184, 186 can be provided in the lower stem 42 and tibial component 40. Optionally, a compliant member 90 and/or dampener may be provided to add compliance to the internal brace in a manner like described above.

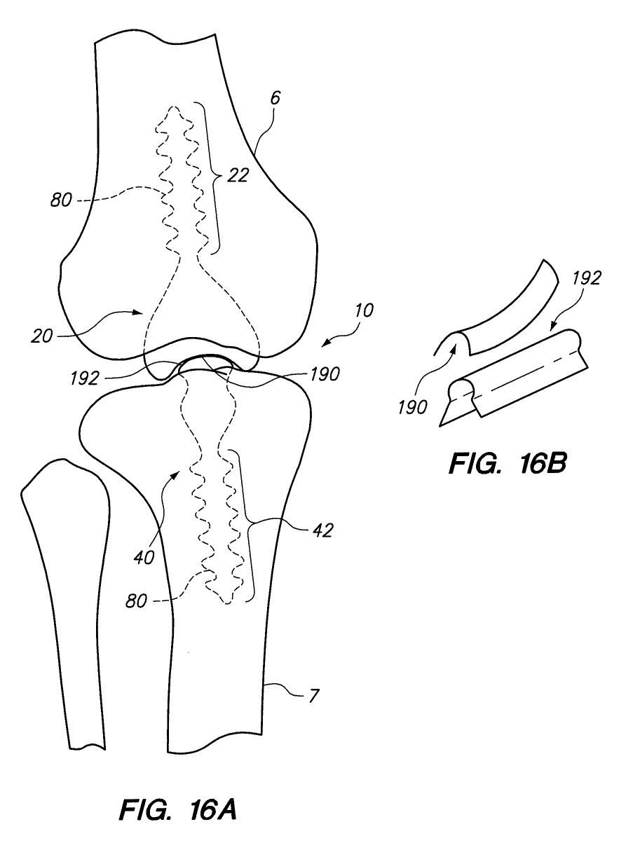

FIG. 16A illustrates an internal brace 10 having been implanted intramedullarly in the femur 6 and tibia 7. In this embodiment, the stem portions 22 and 42 are substantially rod-shaped and function like the shaft of a hip implant, for example, where they are inserted into the medullary canal of the femur or tibia, respectively, and are anchored by an interference fit. Additionally, one or more osteoinduction features 80 may be provided on the surfaces of the shafts 22, 42 to encourage bone ingrowth. Thus, the femoral and tibial components, as implanted, provide contact surfaces 190 and 192 in the center of the knee joint which contact each other and distract the femur 6 and tibia 7. The femoral contact surface 190 may have an elongated (along the anterior to posterior direction) concave saddle shape, as illustrated in FIG. 16B and the tibial contact surface 192 may be convex in the medial-lateral direction to correspond to the concave shape of the contact surface 190 in the medial-lateral direction, but straight (flat) along the anterior to posterior direction.

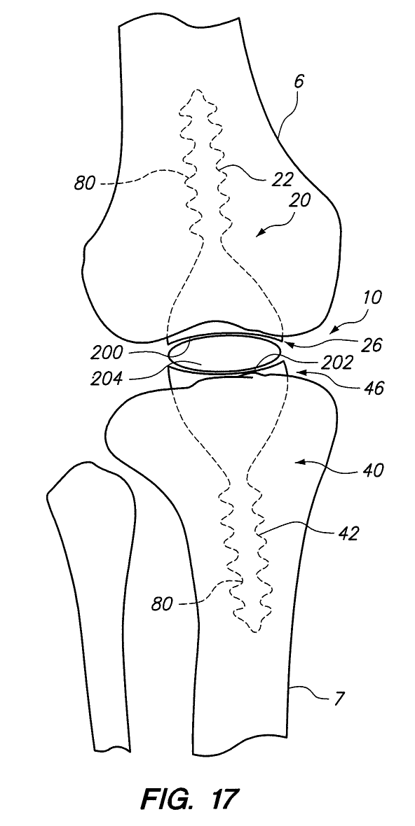

FIG. 17 illustrates another embodiment of an internal brace 10 having been implanted intramedullarly in the femur 6 and tibia 7. In this embodiment, like the embodiment of FIG. 16A, the stem portions 22 and 42 are substantially rod-shaped and function like the shaft of a hip implant, for example, where they are inserted into the medullary canal of the femur or tibia, respectively and are anchored by an interference fit. Additionally, one or more osteoinduction features 80 may be provided on the surfaces of the shafts 22, 42 to encourage bone ingrowth. Portions 26 and 46 of the femoral and tibial components 20 and 40, as implanted, provide contact surfaces 200 and 202 in the center of the knee joint. Contact surfaces 200 and 202 are separate bearing surfaces, each of which interacts with one of opposite bearing surfaces provided on intermediate joint member 204. Intermediate joint member 204 may be a ball joint or may have an oval or elliptical cross section like that shown in FIG. 17, and may be rigid or compliant. The contact surfaces 200 and 202 are concave to generally follow the curvature of the opposing surfaces of the intermediate joint member 204.

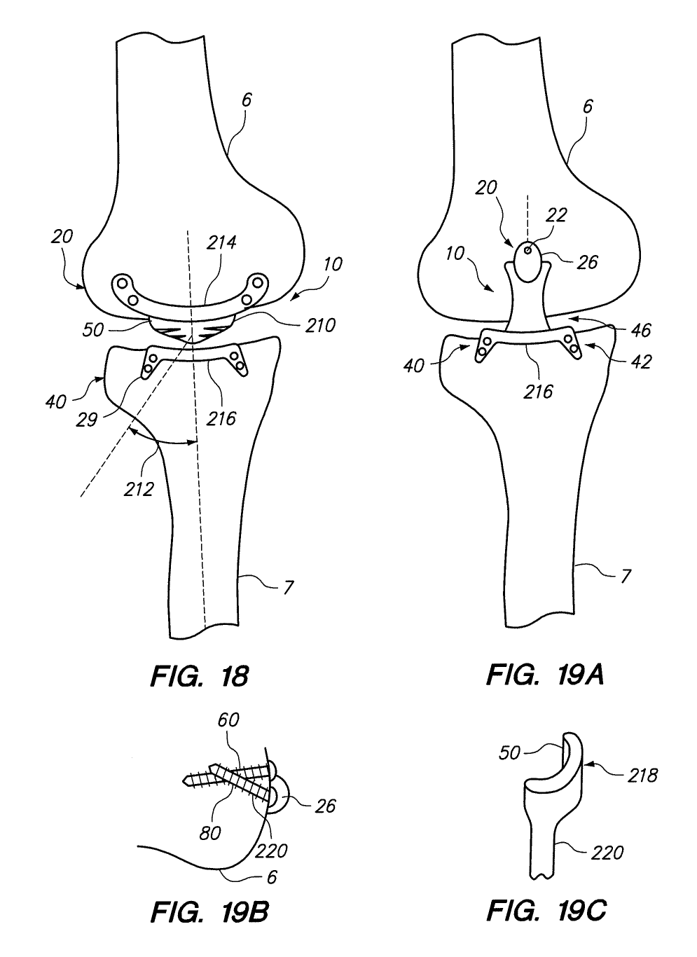

FIG. 18 illustrates another embodiment of an internal brace 10 in which the contact surfaces 29 and 50 contact one another to distract the femur 6 and tibia 7 by a predetermined amount. As in previous embodiments, the shape of the contact surface 29 relative to the contact surface 50 is such that the surfaces 29 and 50 can allow some relative axial rotation between the femur 6 and the tibia 7 during the motions carried out during a gait cycle. Additionally, the shapes and/or dimensions of the surfaces 29 and 50 may be such that they provide distraction/support over only a predetermined portion of the gait cycle. As shown, contact surfaces 29, 50 contact one another only through about the angle 212 shown, which is this example is from about 0 degrees (gait cycle in extension, as shown) to about 45 degrees. Of course, this range can be varied, as noted. Also, the amount of distraction provided over that portion that support is provided can be varied by forming support surface 29 and/or support surface 50 as a cam surface, the radius of curvature of which varies as it is rotated against the opposite surface in the anterior-posterior direction. As shown, surface 29 is a convex surface and surface 29 is flat or only slightly concave so that it does not prevent relative axial rotation between the femur 6 and the tibia 7 during motion (gait cycle).

The lower portion 28 of the femoral component 20 includes cuts 210 that are oriented transverse to the longitudinal axis of the femur 6 when the femoral component is installed thereto. As shown in FIG. 18, cuts 210 are substantially perpendicular to the longitudinal axis of the femur 6. Cuts 210 allow flexion and/or compression of the component 20, so that the distance between the contact surface 29 and the distal end of the femur varies, providing some compliance to the system during walking or running.

One or both of the upper and lower portions 20, 40 can be provided as low profile components. In the example shown, both components 20, 40 are low profile. Each component lacks the stem that is provided with some earlier embodiments. Each component has a recess 214, 216 respectively, that provides clearance for the medial collateral ligament (FIG. 18 shows device 10 installed to the medial side) as it inserts above recess 214 and below recess 216.

The center of rotation, or “pivot point” of the knee joint, about which the tibia 7 and femur 6 rotate during flexion and extension movements of the knee joint is not at the contact surfaces between the femur 6 and tibia 7, but is located superiorly thereof and somewhat anterior of the longitudinal axis of the femur 6. FIGS. 19A-19C illustrate an embodiment of an internal brace 10 in which relative rotation of the components occurs superiorly of the knee joint, preferably near or at the center of rotation of the knee joint. As shown, the upper component 20 comprises a nub 26 that functions as a bearing surface. Typically nub 26 has a spherical surface and functions like a ball joint. A tapered post 220 extends from nub 26 and is configured to be driven into a hole drilled into the femur 6 to provide a compression fit. Post 220 may optionally be provided with one or more osteointegration features 80 of a type described above. The upper portion 22 of femoral component 20 extends from nub 26 and provides an opening through which a screw (locking screws 60, compression screw 64 and/or bicortical screw 62) can be torqued into the femur 6 to further secure the nub 26, and also prevent rotation of the nub 26 relative to the femur 6, see the partial sectional view of FIG. 19B.

The tibial component 40 in this embodiment includes recess 216 to provide clearance for the medial collateral ligament therebelow. The upper portion 46 of the tibial component 40 spans the knee joint when installed as shown in FIG. 19A, extending from the base 42 of the tibial component that is fixed to the tibia 7, across the knee joint and making contact with nub 26 which is fixed to the femur 6. The upper end portion of upper portion 46, which includes contact surface 50 is configured as a cup form 218 (see the partial view of FIG. 19C), which provides a concave contact surface 50 that interfaces with the contact surface of nub 26. The shaft portion 220 of upper portion 46, as shown, is rigid, but optionally, can be modified to provide some vertical compliance.

In use, internal brace 10 provides a predetermined amount of distraction between the femur 6 and the tibia 7, and allows relative axial rotation between the femur 6 and the tibia 7 during the gait cycle. As with previous embodiments, the surface of nub 26 and/or surface 50 of component 218 can be modified to perform like a cam so that the amount of distraction and/or amount of load sharing can be varied at different angles of the gait cycle.

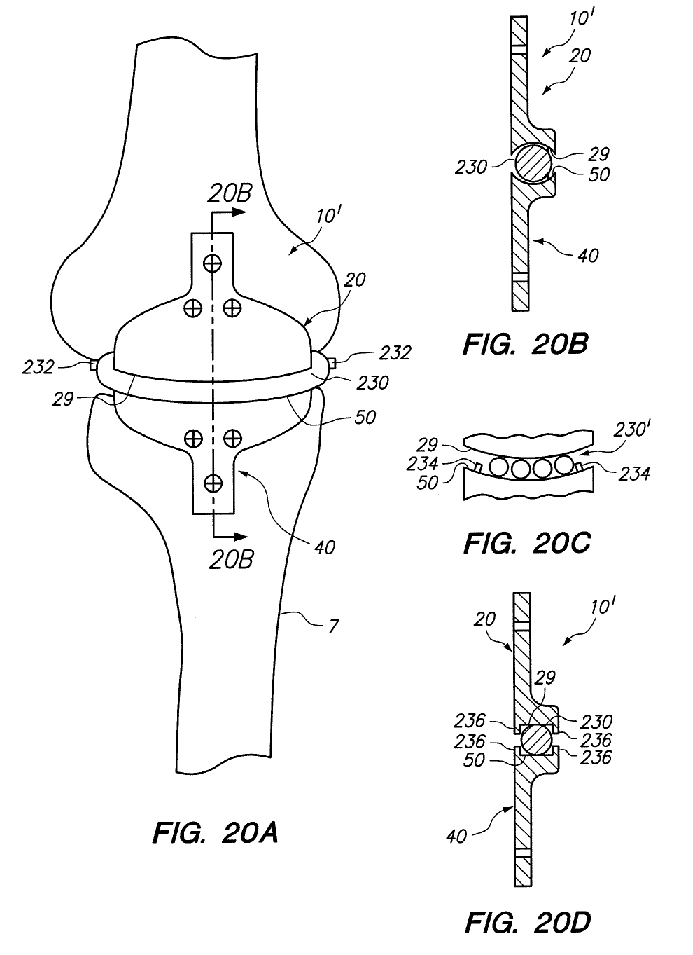

FIG. 20A illustrates an embodiment of a brace 10′ that can be attached medially or laterally (or one brace attached medially and one brace attached laterally) to the femur 6 and tibia 7. As shown, the brace is attached to the medial side. In this embodiment, both contact surfaces 29 and 50 are concave in the medial-lateral direction, while one of the surfaces is convex and one is concave in the anterior-posterior direction. As shown, surface 29 is convex in the anterior posterior direction and surface 50 is concave in the anterior-posterior direction. One of surfaces 29, 50 (surface 50, in the example shown, although it may alternatively be surface 29 if core 230 is attached to the tibial portion) articulates and articulate over a core 230 that may be made of metal, ceramic hard, lubricious polymer, or other hard material, or which may be made from a compliant material. In any case, core 230 is typically softer than the surfaces 29,50 and is therefore the component that wears during use. Accordingly, core 230 is replaceable, so that after a certain amount of wear, or if there is a malfunction, core 230 can be removed and replaced with a new core 230. Core 230 is removably attached to one of upper (femoral) component 20 and lower (tibial) component 40 (as shown, core 230 is attached to upper component 20) via attachment features 232, which may be screws, or core 230 may be provided with holes that fit over pegs extending from the upper or lower portion 20,40 that it is attached to, or other alternative attachment feature that fixes the core 230 to the upper or lower portion 20,40 while allowing it to be removed and replaced. FIG. 20B shows a cross sectional partial view of the device 10′ of FIG. 20A taken along line 20B-20B that shows the interrelationship between the surfaces 29 and 50 relative to core 230. Alternatively, core 230 may be formed as one or more ball bearings, as schematically illustrated in FIG. 20C. In this case, one of surfaces 29, 50 may be provided with stops 234 that prevent ball bearings 230′ from escaping from the anterior or posterior end of the surface. Accordingly, bearings 230 are never exposed beyond an edge of either surface 29 or surface 50. In any of the embodiments of FIGS. 20A-20C, one of the contact surfaces 29, 50 may have a larger radius of curvature in the medial-lateral direction than the other to allow for rotational slippage, to allow relative axial rotation between the femur 6 and tibia 7 during motions performed over the course of the gait cycle. Further alternatively, surfaces 29, 50 may be flat in the medial lateral direction and optionally may be provided with edges 236 that deter malalignment of the components 20, 40, as schematically illustrated in the sectional illustration of FIG. 20D.

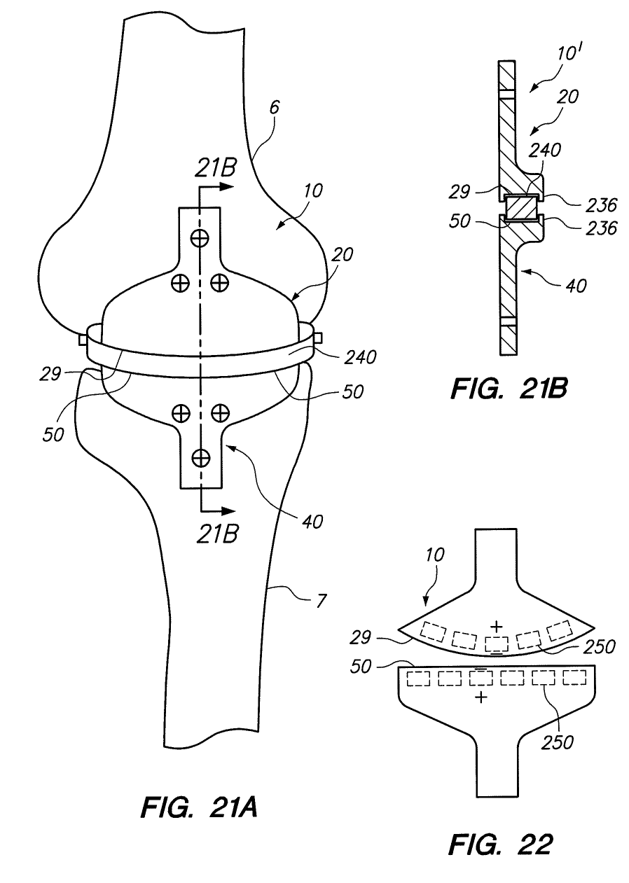

FIG. 21A illustrates a variant of the brace of FIG. 20A, which is installed similarly to and functions similarly to the brace 10′ of FIG. 20A. However, in this embodiment, surface 29 and 50 are flat in the medial-lateral direction like the embodiment of FIG. 20D. Unlike the embodiment of FIG. 20D, core 240, is not spherical or otherwise round in cross section, but has flat surfaces in the anterior-medial direction that interface with the surfaces 29 and 50, as illustrated in the sectional view of FIG. 21B. Core 240 may be replaceable and may be made from any of the same materials as core 230.

FIG. 22 illustrates a feature that is shown with regard to one particular embodiment of a brace, but which may be incorporated into any other embodiment described herein as well. When the contact surfaces 29, 50 of the brace are made of non-magnetizable materials, magnets 250 may be implanted in the brace to create a repulsion to reduce the frictional forces experienced by the contact surfaces 29, 50. By aligning magnets 250 to have like poles of the opposing magnets adjacent one another, this provide a repulsive force that reduces the amount of contact force between the surfaces 29,50 that would otherwise be realized. Magnets may be provided to produce repulsive magnetic forces of sufficient magnitude to repel the contact surfaces 29,50 such that there is no physical contact between surfaces 29, 50. Typically however, magnets 250 are provided to reduce the load applied between the contact surfaces 29, 50 although they still make physical contact with one another and therefore bear a reduced load. Further, the strengths of various pairs of opposing magnets 250 and/or the distances between opposing magnets in the various pairs can be designed to customize the amount of unloading at various portions of the gait cycle to provide a customized joint unloading curve tailored to the specific characteristics of the knee joint of the individual into which it is being implanted.

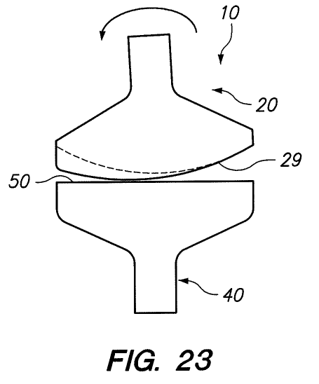

Alternative or in addition to adjusting the amount of load carried by brace 10 by altering the relative location of the upper portion as fixed to the femur and lower portion as fixed to the tibia to customize the amount of the gait cycle during which the knee joint is supported and/or the relative amount of support provided in various portions of (or all) of the gait cycle that is supported, the contour of the interactive surfaces between the upper and lower portions may be customized to vary the load taken on by the device 10 along various portions of the gait cycle. This contour may be customized by customizing the shape of a bearing member between surfaces 29 and 50, or by altering the surfaces of one or both of surfaces 29 and 50. FIG. 23 illustrates one example where surface 29 has been provided with a cam surface in the anterior posterior direction (right to left in FIG. 23. Accordingly, as upper component 20 rotates relative to lower component 40 in the direction of the arrow shown, the radius of curvature of the portion of surface 29 (dotted line shows constant radius of curvature) that contacts surface 50 increases as the gait cycle move from extension (shown) to flexion. This increases the distraction between the femur 6 and tibia 7 and/or increases the load born by brace 10.

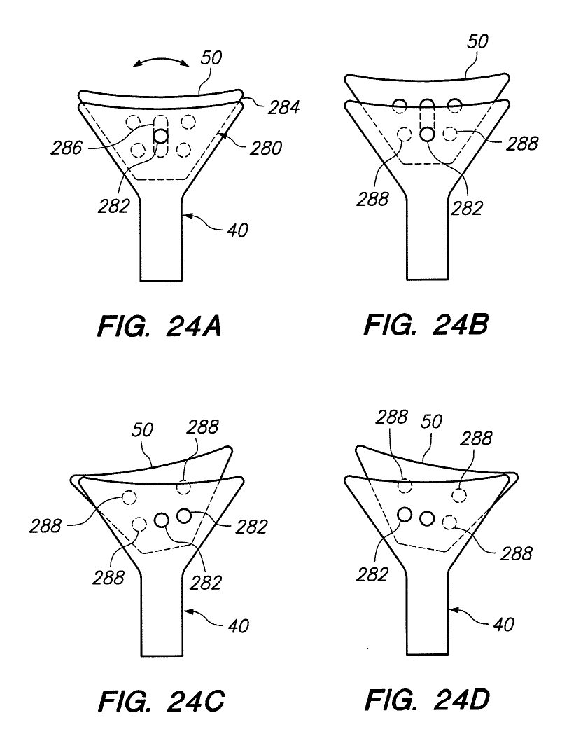

FIGS. 24A-24D illustrate an embodiment wherein device 10 is axially adjustable to uniformly vary the amount of distraction over the entire gait cycle, without the need to reposition either the upper portion or lower portion anchoring locations to the femur 6 and tibia 7. Additionally, FIGS. 24A-24D illustrates an embodiment where the relative amounts of load can be varied over the gait cycle, without the need to move the anchoring locations of the upper and lower portions 20, 40. As shown in FIGS. 24A-24D, adjustment mechanism 280 is provided in the lower portion 40 to provide adjustability to the brace 10 that lower portion 40 forms a part of Alternatively, adjustment mechanism 280 could be provided in the upper portion in the same way.

Adjustment mechanism 280 includes at least one locking member 282, such as a screw, bolt, clamp or other releasable locking feature that can be actuated to lock the adjustable portion 284 that includes the surface 50 relative to the remainder of the lower portion. When unlocked, portion 284 is axially slidable relative to the remainder of lower portion 40. Additionally, when unlocked, portion 284 is rotatable relative to the main body of the lower portion 40 about a limited range of rotation in the directions of the rotational arrows shown in FIG. 24A, e.g., about an axis extending generally in the medial-lateral direction. At least one slot 286 may be provided in portion 284 in which locking feature 282 can slide when in an unlocked configuration, to adjust the axial length of the component 40, as illustrated in FIG. 24B, where the axial length has been increased. Locking feature 282 can be locked, such as by torquing down the screw or bolt against a nut on the opposite side of slot to maintain this adjusted axial length.

Additionally, portion 284 can rotate about locking feature 282, as illustrated in the adjustment positions shown in FIGS. 24C and 24D. Accordingly, adjustments can be made to increase distraction during extension, relative to the amount of distraction provided toward the end of the extension cycle (e.g., see FIG. 24C) to decrease distraction during extension, relative to the amount of distraction provided toward the end of the extension cycle (e.g., see FIG. 24D), by raising or lowering one end of surface 50 relative to the other end. The angular orientation of surface 50 is continuously adjustable to all orientations between the orientations at the end points of the rotational travel of portion 280. Alternatively or additionally, additional holes or slots may be provided in portion 284 in predetermined locations such that they line up with holes in the main body portion of lower portion 40 (or upper portion 20) when the portion 280 has been rotated to an orientation defining a predetermined loading pattern (e.g., predetermined amounts of distraction along the gait cycle having been predetermined). For example, in FIG. 24C, an additional locking feature 282 has been inserted into an aligned opening 288, thereby further securing the mechanism to prevent if from rotation, and to confirm that the surface 50 has been oriented to provide a desired loading profile over the gait cycle. Not that in FIG. 24D, the location where the opening 288 aligns and into which the additional locking feature is placed is different than in FIG. 24C.

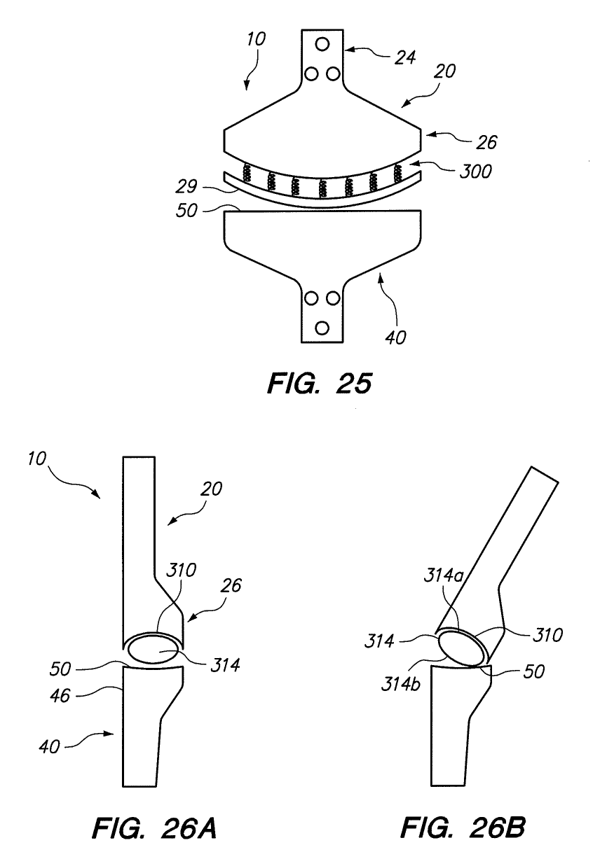

FIG. 25 illustrates an internal brace 10 in which a compliant feature 300 is provided in one of the portions in the brace. In the example shown, compliant feature is provided in the 26 of the femoral component, between surface 29 and the transition to the upper stem portion 24. Alternatively, compliant feature 300 could be similarly installed in the tibial component 40. As shown, compliant member 300 comprises a plurality of coil springs 302 interconnecting the contact member having the contact surface 29 with the remainder of the lower portion 26 and distributed over the space therebetween. Alternative compliant members 302 may be substituted, such as leaf springs, gas filled cylinders, a compliant material having either continuous or variable compliance along its length in the anterior-posterior direction, etc. A plurality of the compliant member 302 extend in the anterior-posterior direction along the portions that they connect. The stiffness of the individual compliant members can be varied to vary the amount of load absorption carried by the brace at different locations over the gait cycle. As another consideration, the area of contact between surfaces 29 and 50 can vary over the course of the gait cycle. Accordingly, the stiffnesses of the complaint members 302 can be varied along the anterior-posterior direction to compensate for the variation in contact area, so as to maintain the same amount of load support (e.g., force per unit area) over the gait cycle if desired. Further adjustability can be provided, for example, by combining with the mechanism of FIGS. 24A-24C, wherein the compliant feature would be installed in portion 280, between the contact surface and locking feature 282.

FIGS. 26A-26B illustrate an embodiment of an internal brace 10 configured to be implanted against the medial or lateral side of a knee joint. As in previously described embodiments, one or more osteoinduction features 80 may be provided on the bone-contacting surfaces of upper and lower portions 20, 40 to encourage bone ingrowth. Portions 26 and 46 of the femoral and tibial components 20 and 40, as implanted, provide contact surfaces 310 and 50 as shown in FIG. 16A with brace 10 oriented as it would be when attached to the knee joint in extension. Contact surfaces 310 and 50 are separate bearing surfaces, each of which interacts with one of opposite bearing surfaces provided on intermediate joint member 314. Alternatively, intermediate joint member 314 could be made integral with surface 310, so that there would no longer be an intermediate joint member, but only contact and movement between the lower surface of 314 and surface 50. In either case, at least the lower surface 314b may have elliptical curvature or spherical curvature. When provided with elliptical curvature, the elliptical shaped curve extends in the anterior-posterior direction (left to right in FIGS. 26A-26B) so that the intermediate joint member 314 provides a greater range over which the components 20,40 may be flexed while still maintaining contact with the intermediate joint member, relative to the range provided by a spherical surface, or ball-shaped intermediate joint member 314. As shown, member 314 is elliptical-shaped, having elliptical curvature over both the upper and lower surfaces 314a, 314b. Intermediate joint member 314 may thus be a ball joint or may have an oval or elliptical cross section as described. Intermediate joint member 314 may be rigid (i.e., non-yielding under the loads it experiences during use) or compliant, so that it deforms and absorbs at least part of the load applied to it during use. The contact surface 310 is concave to generally follow the curvature of the opposing surface of the intermediate joint member 314 and contact surface 50 is flat or nearly flat.

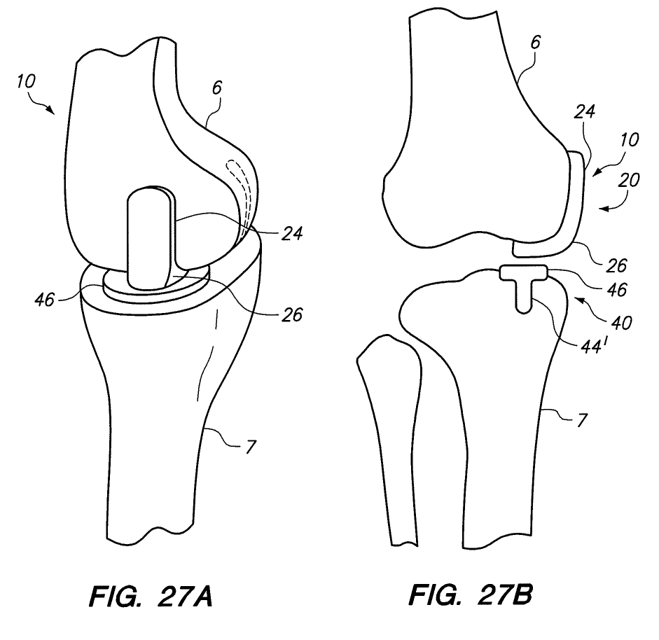

FIGS. 27A-27B show a side view and an anterior view, respectively, of a device 10 employing an intra-articular tibial component 40. In this embodiment, the femoral or upper component 20 is like that described in previous embodiments in that elongated stem 24 is mounted extra-articularly, outside of the joint space and component 26 is the same as those in described in previous embodiments. However, lower or tibial component 40 includes an elongated stem 44′ that is implanted intra-articularly, in the tibial tray. Component 46 can be configured to function in any of the same manners described with regard to previous embodiments.

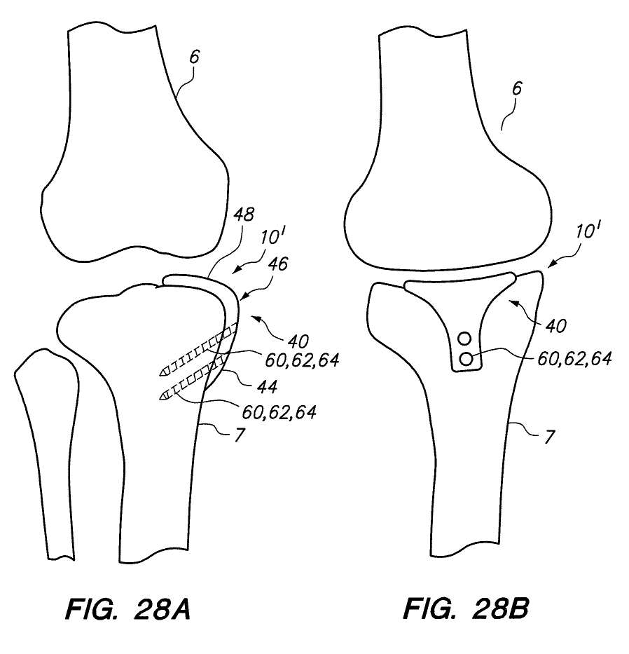

FIGS. 28A-28B show an anterior view and a side view, respectively, of a single component brace 10′. In this embodiment, the single component is a lower component 40. Alternatively, a single component brace 10′ can be constructed from an upper component 20, depending upon various factors, typically including the condition/amount of damage or disease of the upper and lower natural load bearing contact surfaces. In FIGS. 28A-28B, component 46 and contact surface 48 contacts and interacts with the opposing natural contact surface on the tibia, and, depending on the thickness of portion 48 may distract that portion of the joint not overlain by portion 48. Portion 48 may be made as a short wedge portion, like in FIG. 10B, for example, so as not to overlie the tibial meniscus and so as to distract the joint on the side that the brace 10′ is implanted over at least a portion of the range of motion of the joint.

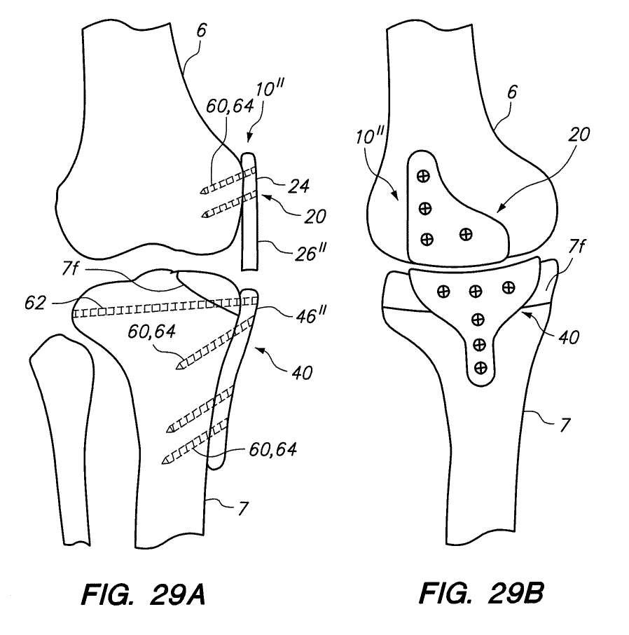

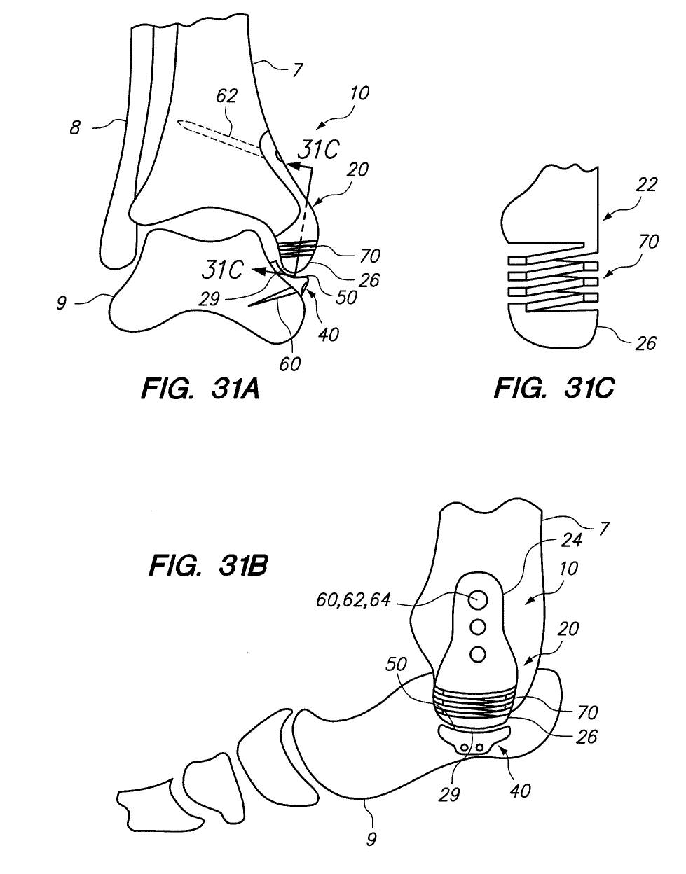

FIGS. 29A-29B show an anterior view and a side view, respectively, of a component brace 10″ configured for treatment of trauma. In this example, the tibial condyle has been fractured at 7f. However, brace 10″ is not limited to treatment of fractured tibial condyles, but can be used similarly for femoral condyle fractures, other fractures and/or other traumas to the knee joint, and can be configured for treatment of other joints having undergone trauma. In the example of FIGS. 29A-29B. the upper and lower components 26″, 46″ including the contact surfaces that contact one another to distract the bones that form the joints, are located entirely outside of the joint space. This is important in this instance so as not to interfere with the traumatized tissue, to allow it to heal without having to perform any weight bearing or any interaction with the contact surfaces of brace 10″. A bicortical screw 62 is shown extending through the fractured bone portion to replace it into its natural position and hold it in place during healing, while at the same time mounting a portion of the lower portion 40 to the tibia. Additional locking screws 60, bicortical screws 62 or compression screws 64, or some combination thereof can be inserted through the lower portion 40 and/or fractured bone portion as shown. Alternative to what is shown in FIG. 31A, the fracture bone portion may be fixed by one or more dedicated screws, 60,62,64 that do/does not pass through lower portion 40 and therefore is/are not used to also mount the lower portion. This decouples stresses applied to the lower portion during use of brace 10″ and movement of the joint (i.e., the gait cycle), allowing healing to proceed uninterrupted by these forces on the brace. However, it may be preferred to use the arrangement of FIG. 29A as the cyclical loading of the traumatized bone portion may help in remodeling the bone during healing. The upper portion 20 can be mounted in any of the same ways described above with regard to upper portions 20. The contact surfaces of portions 26″ and 46″, as noted, are completely outside the joint and these portions can be configured to contact one another so as to distract the joint through all of the range of motion of the joint. After a predetermined period of healing, portions 26″ and 46″ may be removable to alter the amount of distraction, so as to allow some load sharing by the natural joint in any of the manners described above with regard to FIGS. 10A-10D and 11A, for example.

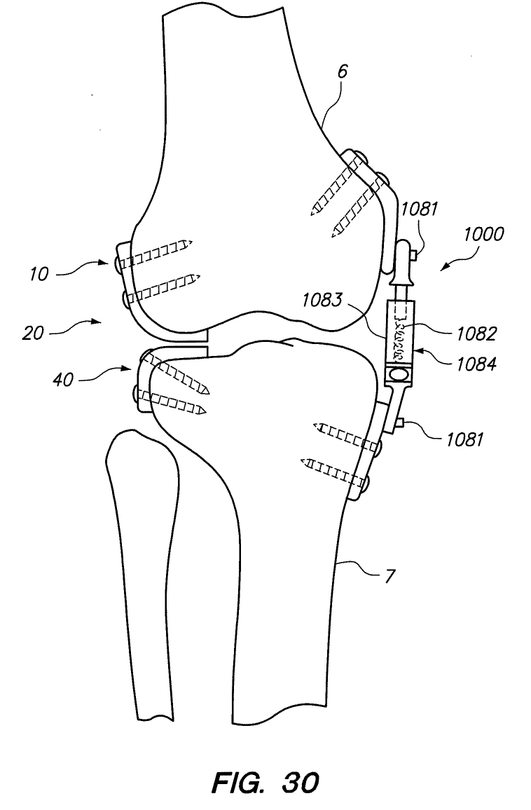

FIG. 30 illustrates an internal brace 10 according to the present invention implanted on the lateral side of the knee joint, in combination with an energy manipulation system 1000 implanted on the medial side of the knee joint. Articulating surfaces 1081 of the energy manipulation system allow multiple degrees of freedom between the base anchors and the energy absorber assembly 1084, including the energy absorbing structure 1082 configured within a stabilizer, such as sliding sleeve 1083. This energy absorbing structure shares and absorbs energy between body parts, in this instance between the femur 6 and the tibia 7. During use, any load transfer that may occur to the medial side of the knee joint when the lateral side is distracted by brace 10 is absorbed by energy manipulation system 1000 on the medial side of the knee joint. Preferably, brace 10 and energy manipulation system 1000 are designed to balance the load between lateral and medial sides. It is noted here that an opposite configuration is also possible, i.e., where energy manipulation system is implanted on the lateral side of a knee joint and internal brace 10 is implanted on the medial side of the knee joint. It is further noted that, in these combinations, just as in other combinations described above, and in uses of single internal braces described above, an energy manipulation system 1000 and internal brace 10 may be implanted on opposite sides of a joint in the body other than the knee joint. Further details of energy manipulation systems usable as described herein can be found in co-pending, commonly-owned application Ser. No. 11/743,605 filed May 2, 2007 and titled “Extra-Articular Implantable Mechanical Energy Absorbing System” and in co-pending, commonly-owned application Ser. No. 11/755,149 filed Jul. 9, 2007 ad titled “Extra-Articular Implantable Mechanical Energy Absorbing System and Implantation Method”. Both application Ser. No. 11/743,605 and application Ser. No. 11/755,149 are hereby incorporated herein, in their entireties, by reference thereto.

FIGS. 31A and 31B show an anterior-posterior view and a lateral view of an internal brace implanted to an ankle joint. The only bones shown in FIG. 31A are the tibia 7 (partial), fibula 8 (partial) and talus 9, while the lateral view of FIG. 31B illustrates additional bones of the foot anterior to the talus 9 and the fibula 8 is not visible. Upper portion 20 is anchored to the tibia via one or more fasteners, such as screws, which may be locking screws 60, bicortical screws 62 or compression screws 64, or some combination thereof. Likewise, lower portion 40 is anchored to the talus 9 via one or more fasteners, such as screws, which may be locking screws 60, bicortical screws 62 or compression screws 64, or some combination thereof.

FIG. 31C illustrates a sectional view of a portion of the upper component 20 taken along line 31C-31C in FIG. 31A. In this example, compliant member 70 is a single piece coil spring integrally formed into upper portion by machining. As in earlier described embodiments, the type as well as location of compliant member 70 may vary.

In descriptions provided herein regarding distraction and modification of distraction forces, it is noted that the devices 10 described herein can also be configured to alter the joint reaction force without distracting the joint, by applying a force, which if large enough, would cause distraction, but by keeping the applied force below a limit force that begins to cause distraction. Accordingly, the contacting joint surfaces are not separated by this approach, but the load experienced by the contacting joint surfaces is reduced by the brace, over one or more locations of the range of motion of the joint (up to all locations). Thus, the brace in this situation is a load sharing brace, rather than relieving all of the load from the compartment by distracting the femur and tibia on that side.

When using a bicompartmental approach, at least one of the devices 10 (lateral and/or medial) may be adjustable as to location about which it rotates, amount of load taken up at different positions along the gait cycle, amount of distraction, if any, at different positions along the gait cycle, and/or amount of compliance, if any, provided, etc.

A device 10 may be installed on a joint such that the positioning of the device or linkage to screws into the bones that the device is attached to can be used to apply torque to the joint, with or without also applying distraction.

The devices described herein may be used as permanent implants, or may be configured to be implanted only temporarily, and then later removed.

The present invention provides, in combination, an internal brace configured to be implanted on one side of a joint and an energy manipulation system configured to be implanted on an opposite side of the joint, said internal brace comprising: a first component for attachment to a distal end portion of a first bone of a patient, said first component including a first upper portion configured to be fixed to the first bone and a first lower portion tapering from said first upper portion and including a first bearing surface; a second component for attachment to a proximal end portion of a second bone of the patient, wherein the joint is formed between the distal end portion of the first bone and the proximal end portion of the second bone, said second component including a second lower portion configured to be fixed to the second bone and a second upper portion tapering from said second lower portion and including a second bearing surface; wherein said first and second bearing surfaces are configured to allow relative rotation between said first and second bones; and said energy manipulation system comprising: a first attachment structure configured to be attached to the first bone; a second attachment structure configured to be attached to the second bone; and an energy absorbing member attached to the first attachment structure and the second attachment structure.

In at least one embodiment, the first and second bearing surfaces are configured to further allow at least one of: relative translation between said first and second bones along a direction; and at least a second degree of freedom of relative rotation between the first and second bones.

A method to reduce pain is provided, including: implanting an internal brace on one side of a natural joint to reduce energy transferred through the natural joint; and implanting an energy absorber on an opposite side of the natural joint in a manner to bear at least a portion of a load transfer that may occur from said one side of the natural joint as the internal brace functions to reduce energy transferred through the joint.

In at least one embodiment, the internal brace distracts the natural joint on said one side over at least a portion of the cycle of natural movement of the joint.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention.

We claim:

1. An internal brace for providing support to a joint, said brace comprising:
a first component for attachment to a distal end portion of a first bone of a patient, said first component including a first upper portion configured to be fixed to the first bone and a first lower portion tapering from said first upper portion and including a first bearing surface;
a second component for attachment to a proximal end portion of a second bone of the patient, wherein a joint is formed between the distal end portion of the first bone and the proximal end portion of the second bone, said second component including a second lower portion configured to be fixed to the second bone and a second upper portion tapering from said second lower portion and including a second bearing surface;
wherein said first and second bearing surfaces are configured to allow relative rotation between said first and second bones and to allow at least one of: relative translation between said first and second bones along a direction; and at least a second degree of freedom of relative rotation between the first and second bones.

2. The brace claim of claim 1, wherein the first and second bearing surfaces are configured to allow said relative translation along an anterior-posterior direction.

3. The brace of claim 1, wherein the first and second bearing surfaces articulate against one another.

4. The brace of claim 1, wherein the first and second bearing surfaces each articulate with a third bearing member.

5. The brace of claim 1, wherein the brace is configured to distract at least one side of the joint, so that the at least one side does not bear a load during motions of the joint.

6. The brace of claim 1, wherein the brace is configured to share load with at least one side of the joint, so that the at least one side of the joint bears a reduced load during motions of the joint.

7. The brace of claim 1, wherein the bearing surfaces support a load during only a portion of the full range of motion of the joint.

8. The brace of claim 1, wherein the bearing surfaces are configured to support varying amounts of load over varying portions of the full range of motion of the joint.

9. The brace of claim 1, wherein the brace is adjustable to vary at least one of: a location about which at least one of said bearing surfaces rotates; an amount of load taken up at different positions along the range of motion of the joint; an amount of distraction at different positions along the range of motion of the joint, and amount of compliance provided by said brace.

10. The brace of claim 1, wherein the first lower portion and said upper portion in combination form a wedge for distracting the joint.

11. The brace of claim 1, wherein a pair of internal braces are adapted to be placed on both sides of a patient's knee joint.

12. The brace of claim 1, wherein at least one compliant member is configured to allow axial movement between the first and second bones.

13. The brace of claim 1, wherein the brace is configured to support a knee joint, wherein said first component comprises a femoral component and said first lower portion tapers outwardly into a condylar protrusion, said first bearing surface comprising a lower surface of said condylar protrusion, wherein the upper surface of the condylar protrusion is adapted to conform to the condyle, and wherein said first upper portion comprises a first inner surface configured to be attached to the femur and an outer surface that is external of the femur when said first inner surface is attached to the femur, and wherein said second component comprises a tibial component and said second upper portion tapers outwardly from said second lower portion into an upper tray comprising said second bearing surface for engaging the first bearing surface of the condylar protrusion, and wherein said second lower portion comprises a second inner surface configured to be attached to the tibia and a second lower portion outer surface that is external of the tibia when said second inner surface of the second lower portion is attached to the tibia.

14. The brace of claim 1, wherein the femoral and tibial components are adapted to be attached to the medial side of the patient's knee, said condylar protrusion and said upper tray in combination form a wedge adapted to fit into at least a portion of the meniscal space in the patient's medial joint.

15. The brace of claim 1, wherein the femoral and tibial components are configured to be attached to the patient's femur and tibia, respectively, without substantially removing or replacing articular cartilage and with the first bearing surface engaging the second bearing surface, the condylar protrusion and the upper tray adapted to be positioned partially in the joint between the patient's intact femur and tibia and functioning to distract the joint.

16. The brace of claim 1, wherein the brace is configured to support an ankle joint.

17. A method for treating a joint including:
providing an internal brace including a first component for attachment to a distal end portion of a first bone of a patient, said first component including a first upper portion configured to be fixed to the first bone and a first lower portion tapering from said first upper portion and including a first bearing surface, and a second component for attachment to a proximal end portion of a second bone of the patient, wherein the joint is formed between the distal end portion of the first bone and the proximal end portion of the second bone, said second component including a second lower portion configured to be fixed to the second bone and a second upper portion tapering from said second lower portion and including a second bearing surface, and at least one compliant member to allow movement between the first and second bones;
attaching the first upper portion of the first component to distal end portion of the patient's first bone; and
attaching the second component to the proximal end portion of the patient's second bone such that the first bearing surface engages the second bearing surface without substantially removing or replacing articular cartilage in the joint, to support the joint, wherein said first and second bearing surfaces are configured to allow relative rotation between said first and second bones and to allow at least one of: relative translation between said first and second bones along a direction; and at least a second degree of freedom of relative rotation between the first and second bones.

18. The method of claim 17, wherein the first and second bearing surfaces are configured to allow said relative translation along an anterior-posterior direction.

19. The method of claim 17, wherein the brace is configured to support a knee joint, wherein said first component comprises a femoral component and said first lower portion tapers outwardly into a condylar protrusion, said first bearing surface comprising a lower surface of said condylar protrusion, wherein the upper surface of the condylar protrusion is adapted to conform to the condyle, and wherein said first upper portion comprises a first inner surface configured to be attached to the femur and an outer surface that is external of the femur when said first inner surface is attached to the femur, and wherein said second component comprises a tibial components and said second upper portion tapers outwardly from said second lower portion into an upper tray comprising said second bearing surface for engaging the first bearing surface of the condylar, and wherein said second lower portion comprises a second inner surface configured to be attached to the tibia and a second lower portion outer surface that is external of the tibia when said second inner surface of the second lower portion is attached to the tibia.

20. The method of claim 19, wherein the condylar protrusion and upper tray, in combination, form a wedge distracting said joint.

21. The method of claim 17, wherein the method further includes attaching an additional internal knee brace, whereby internal knee braces are attached to both the medial and lateral joints of the patient's knee.
